(19) United States
(12) Patent Application Publication
(10) Pub. No.: US 2025/0025175 A1
(43) Pub. Date: Jan. 23, 2025

(54) TISSUE ANCHOR DEPLOYMENT DEVICE, SYSTEMS, AND METHODS

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Collin Shale, Medford, MA (US); Kevin M. Falco, Chelmsford, MA (US); John Prudden, Manchester, MA (US); Luis Alejandro Miranda Cruz, San Francisco (CR); Dennis Colleran, North Attleboro, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/770,279

(22) Filed: Jul. 11, 2024

Related U.S. Application Data
(60) Provisional application No. 63/528,020, filed on Jul. 20, 2023.

(51) Int. Cl. A61B 17/10; A61B 17/00

(52) U.S. Cl. CPC A61B 17/105; A61B 2017/00407

(57) ABSTRACT

A system for delivering tissue anchors into an operative space may include a tissue anchor delivery device including a handle and an elongate shaft, and a tissue anchor magazine separate and detached from the tissue anchor delivery device. The magazine may be configured to be delivered subcutaneously into the operative space separate from the tissue anchor delivery device. Another tissue anchor delivery device may include an elongate shaft having a length from a handle to a distal end of the shaft of at least 10 cm, a tissue anchor magazine coupled to the handle in communication with the shaft, and a tissue anchor advancement mechanism configured to advance a plurality of tissue anchors from the magazine out the distal end of the shaft. The tissue anchor advancement mechanism may be configured to advance only one tissue anchor along the length of the shaft at a time.

### CROSS REFERENCE TO RELATED APPLICATIONS

This application claims the benefit of U.S. Provisional Patent Application Ser. No. 63/528,020 filed on Jul. 20, 2023, the disclosure of which is incorporated herein by reference.

### TECHNICAL FIELD

The present disclosure pertains generally to medical devices and methods of using medical devices. More particularly, the present disclosure relates to medical devices and/or systems, and methods of using the same, for securing a medical implant to tissue in an operative space in a body of a patient

### BACKGROUND

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. Current procedures for treatment of a torn tendon include affixing a biocompatible implant over the torn tendon. There is an ongoing need to deliver and adequately secure medical implants during an arthroscopic procedure in order to treat injuries to the rotator cuff, rotator cuff tendons, or other soft tissue or tendon injuries throughout a body.

### SUMMARY

This disclosure provides design, material, manufacturing method, and use alternatives for medical devices, including system for delivering tissue anchors into an operative space.

A first example is a system for delivering tissue anchors into an operative space in a body of a patient. The system includes a tissue anchor delivery device comprising a handle and an elongate shaft extending distally from the handle. The system also includes a tissue anchor magazine separate and detached from the tissue anchor delivery device. The tissue anchor magazine has a plurality of tissue anchors stored therein. The tissue anchor magazine is configured to be delivered subcutaneously into the operative space separate from the tissue anchor delivery device.

Alternatively or additionally to any of the examples above, in another example, the system includes an implant delivery device including an elongate shaft configured to deliver an implant into the operative space.

Alternatively or additionally to any of the examples above, in another example, the tissue anchor magazine is configured to be detachably coupled to a distal portion of the elongate shaft of the implant delivery device.

Alternatively or additionally to any of the examples above, in another example, the implant delivery device includes a tether coupled to the implant and configured to extend proximally to a free end disposed outside of the operative space after the elongate shaft of the implant delivery device has been removed from the operative space.

Alternatively or additionally to any of the examples above, in another example, the tissue anchor magazine includes a lumen extending therethrough, wherein the lumen is configured to slidably receive the tether therein such that the tissue anchor magazine is advanceable over the tether to the operative space after deployment of the implant.

Alternatively or additionally to any of the examples above, in another example, the tissue anchor magazine is axially securable to the tether within the operative space.

Alternatively or additionally to any of the examples above, in another example, the tissue anchor magazine is configured to be coupled to tissue within the operative space.

Alternatively or additionally to any of the examples above, in another example, the tissue anchor magazine is configured to releasably couple with a delivery handle configured to insert the tissue anchor magazine into the operative space.

Alternatively or additionally to any of the examples above, in another example, the tissue anchor magazine includes an anchoring barb at a distal end thereof.

Alternatively or additionally to any of the examples above, in another example, the delivery handle is configured to retract the anchoring barb into the tissue anchor magazine, thereby decoupling the tissue anchor magazine from tissue within the operative space.

Alternatively or additionally to any of the examples above, in another example, engagement of a distal end of the elongate shaft of the tissue anchor delivery device with the tissue anchor magazine within the operative space is configured to load one tissue anchor of the plurality of tissue anchors onto the tissue anchor delivery device in situ.

Alternatively or additionally to any of the examples above, in another example, each tissue anchor of the plurality of tissue anchors is oriented at an oblique angle relative to a longitudinal axis of the tissue anchor magazine.

Another example is a method of securing an implant to tissue in an operative space in a body of a patient. The method includes advancing the implant into the operative space within an elongate shaft of an implant delivery device and deploying the implant adjacent tissue within the operative space. A tissue anchor magazine is positioned within the operative space. The tissue anchor magazine has a plurality of tissue anchors stored therein. An elongate shaft of a tissue anchor delivery device is advanced into the operative space separately from the tissue anchor magazine. The tissue anchor delivery device comprises an elongate shaft extending distally from a handle. A first tissue anchor is deployed from the elongate shaft of the tissue anchor delivery device into the implant. Thereafter, a distal end of the elongate shaft of the tissue anchor delivery device is engaged with the tissue anchor magazine within the operative space to load a second tissue anchor from the plurality of tissue anchors onto the tissue anchor delivery device in situ. The second tissue anchor is then deployed from the elongate shaft of the tissue anchor delivery device into the implant.

Alternatively or additionally to any of the examples above, in another example, positioning the tissue anchor magazine within the operative space includes attaching the tissue anchor magazine to a distal portion of the elongate shaft of the implant delivery device.

Alternatively or additionally to any of the examples above, in another example, positioning the tissue anchor magazine within the operative space includes securing the tissue anchor magazine to tissue within the operative space.

Alternatively or additionally to any of the examples above, in another example, positioning the tissue anchor magazine within the operative space includes sliding the tissue anchor magazine over a tether coupled to the implant and extending to a free end outside of the operative space.

Alternatively or additionally to any of the examples above, in another example, the method includes securing the tissue anchor magazine to the tether within the operative space.

Alternatively or additionally to any of the examples above, in another example, the method includes removing the tissue anchor magazine from the operative space after securing the implant to tissue.

Another example is a tissue anchor delivery device. The device includes a handle, an elongate shaft extending distally from the handle, a tissue anchor magazine, and a tissue anchor advancement mechanism. The elongate shaft has a length from the handle to a distal end of the elongate shaft of at least 10 cm. The tissue anchor magazine is coupled to the handle in communication with the elongate shaft. The tissue anchor magazine has a plurality of tissue anchors disposed therein. The tissue anchor advancement mechanism is configured to advance the plurality of tissue anchors from the tissue anchor magazine out the distal end of the elongate shaft. The tissue anchor advancement mechanism is configured to advance only one tissue anchor of the plurality of tissue anchors along the length of the elongate shaft at a time.

Alternatively or additionally to any of the examples above, in another example, each actuation of the tissue anchor advancement mechanism advances the only one tissue anchor of the plurality of tissue anchors only a portion of the length of the elongate shaft. Alternatively or additionally to any of the examples above, in another example, the tissue anchor advancement mechanism includes a ratcheting feature.

Alternatively or additionally to any of the examples above, in another example, the tissue anchor advancement mechanism comprises a pusher coiled within the handle, the pusher being configured to advance the only one tissue anchor of the plurality of tissue anchors along the length of the elongate shaft.

Alternatively or additionally to any of the examples above, in another example, the pusher is configured to uncoil as the only one tissue anchor of the plurality of tissue anchors is advanced along the length of the elongate shaft.

Alternatively or additionally to any of the examples above, in another example, the tissue anchor advancement mechanism comprises a plurality of gears disposed within the handle, the plurality of gears being arranged longitudinally relative to each other within the handle.

Alternatively or additionally to any of the examples above, in another example, the tissue anchor advancement mechanism comprises a continuously looped element engaged with the plurality of gears and a pusher coupled to the continuously looped element, the pusher being configured to advance the only one tissue anchor of the plurality of tissue anchors along the length of the elongate shaft.

Alternatively or additionally to any of the examples above, in another example, at least a portion of the pusher is configured to at least partially wrap around the continuously looped element prior to advancing the only one tissue anchor of the plurality of tissue anchors along the length of the elongate shaft and configured to extend away from the continuously looped element after advancing the only one tissue anchor of the plurality of tissue anchors along the length of the elongate shaft.

Alternatively or additionally to any of the examples above, in another example, the tissue anchor advancement mechanism comprises a linear rack engaged with the plurality of gears and a pusher coupled to the linear rack and extending longitudinally therefrom, the pusher being configured to advance the only one tissue anchor of the plurality of tissue anchors along the length of the elongate shaft.

Alternatively or additionally to any of the examples above, in another example, the linear rack is configured to engage multiple different gears of the plurality of gears as the only one tissue anchor of the plurality of tissue anchors is advanced along the length of the elongate shaft.

Alternatively or additionally to any of the examples above, in another example, the tissue anchor magazine is integrally formed with the handle.

Alternatively or additionally to any of the examples above, in another example, the tissue anchor magazine is configured to be removably received within a receptacle formed in the handle.

Alternatively or additionally to any of the examples above, in another example, the handle comprises a trigger configured to actuate the tissue anchor advancement mechanism.

Alternatively or additionally to any of the examples above, in another example, the trigger is pivotably coupled to the handle.

Alternatively or additionally to any of the examples above, in another example, the trigger is slidably coupled to the handle.

Alternatively or additionally to any of the examples above, in another example, the tissue anchor advancement mechanism comprises an automatic retraction apparatus configured to reset the tissue anchor advancement mechanism.

Alternatively or additionally to any of the examples above, in another example, the tissue anchor advancement mechanism comprises a pusher configured to engage the only one tissue anchor of the plurality of tissue anchors.

Alternatively or additionally to any of the examples above, in another example, the pusher comprises a distal face and a bevel extending proximally from the distal face to a bottom surface such that a height of the distal face is less than a thickness of the only one tissue anchor of the plurality of tissue anchors.

Alternatively or additionally to any of the examples above, in another example, the bevel is configured to engage a subsequent tissue anchor of the plurality of tissue anchors and bias remaining tissue anchors of the plurality of tissue anchors away from the only one tissue anchor of the plurality of tissue anchors as the only one tissue anchor of the plurality of tissue anchors is advanced distally.

Another example is a method of securing an implant to tissue in an operative space in a body of a patient. The method includes advancing the implant into the operative space within an elongate shaft of an implant delivery device and deploying the implant adjacent tissue within the operative space. A tissue anchor delivery device is inserted into the operative space. The tissue anchor delivery device includes a handle, an elongate shaft extending distally from the handle, a tissue anchor magazine, and a tissue anchor advancement mechanism. The elongate shaft has a length from the handle to a distal end of the elongate shaft of at least 10 cm. The tissue anchor magazine is coupled to the handle in communication with the elongate shaft. The tissue anchor magazine has a plurality of tissue anchors disposed therein. The tissue anchor advancement mechanism is configured to advance the plurality of tissue anchors from the tissue anchor magazine out the distal end of the elongate shaft. The method includes actuating the tissue anchor advancement mechanism to deploy at least one tissue anchor of the plurality of tissue anchors out the distal end of the elongate shaft into the implant and tissue within the operative space. The tissue anchor advancement mechanism is configured to advance only one tissue anchor of the plurality of tissue anchors along the length of the elongate shaft at a time.

Alternatively or additionally to any of the examples above, in another example, actuating the tissue anchor advancement mechanism comprises extending a pusher from the handle distally into the elongate shaft.

Alternatively or additionally to any of the examples above, in another example, extending the pusher includes extending the pusher to the distal end of the elongate shaft.

Alternatively or additionally to any of the examples above, in another example, the pusher is coiled within the handle, and the pusher is configured to uncoil as the only one tissue anchor of the plurality of tissue anchors is advanced along the length of the elongate shaft.

Alternatively or additionally to any of the examples above, in another example, the tissue anchor advancement mechanism comprises a plurality of gears disposed within the handle, the plurality of gears being arranged longitudinally relative to each other within the handle.

Alternatively or additionally to any of the examples above, in another example, the tissue anchor advancement mechanism comprises a continuously looped element engaged with the plurality of gears and the pusher is coupled to the continuously looped element.

Alternatively or additionally to any of the examples above, in another example, movement of the continuously looped element about the plurality of gears translates the pusher relative to the elongate shaft.

Alternatively or additionally to any of the examples above, in another example, the tissue anchor advancement mechanism comprises a linear rack engaged with the plurality of gears and the pusher is coupled to the linear rack and extends longitudinally therefrom.

Alternatively or additionally to any of the examples above, in another example, actuation of the tissue anchor advancement mechanism rotates the plurality of gears and advances the linear rack along the plurality of gears such that the linear rack engages multiple different gears of the plurality of gears as the only one tissue anchor of the plurality of tissue anchors is advanced along the length of the elongate shaft.

Alternatively or additionally to any of the examples above, in another example, actuating the tissue anchor advancement mechanism comprises engaging a trigger coupled to the handle.

Alternatively or additionally to any of the examples above, in another example, actuating the tissue anchor advancement mechanism comprises engaging a distal face of the pusher against the only one tissue anchor to advance the only one tissue anchor from the tissue anchor magazine into the elongate shaft.

Alternatively or additionally to any of the examples above, in another example, advancing the distal face of the pusher distal of a proximal face of a subsequent tissue anchor of the plurality of tissue anchors as the only one tissue anchor is advanced from the tissue anchor magazine into the elongate shaft biases remaining tissue anchors of the plurality of tissue anchors toward the tissue anchor magazine.

Alternatively or additionally to any of the examples above, in another example, the method includes resetting the tissue anchor advancement mechanism.

Alternatively or additionally to any of the examples above, in another example, the tissue anchor advancement mechanism comprises an automatic retraction apparatus configured to reset the tissue anchor advancement mechanism.

The above summary of some embodiments, aspects, and/or examples is not intended to describe each disclosed embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cutaway view schematically illustrating selected aspects of a shoulder of a patient;

FIG. 2 schematically illustrates selected aspects of a system for delivering tissue anchors and securing an implant to tissue in an operative space;

FIG. 3A is a perspective view illustrating selected aspects of a tissue anchor magazine;

FIG. 3B is a top view illustrating selected aspects of the tissue anchor magazine of FIG. 3A;

FIG. 3C is a front view illustrating selected aspects of the tissue anchor magazine of FIG. 3A;

FIG. 4 schematically illustrates selected aspects of positioning and/or using the tissue anchor magazine of FIGS. 3A-3C in the operative space;

FIGS. 5-7 schematically illustrate selected aspects of delivering tissue anchors using the system of FIG. 4;

FIG. 8A is a perspective view illustrating selected aspects of a tissue anchor magazine;

FIG. 8B is a top view illustrating selected aspects of the tissue anchor magazine of FIG. 8A;

FIG. 8C is a front view illustrating selected aspects of the tissue anchor magazine of FIG. 8A;

FIGS. 9-10 schematically illustrate selected aspects of positioning and/or using the tissue anchor magazine of FIGS. 8A-8C in the operative space;

FIG. 11A is a perspective view illustrating selected aspects of a tissue anchor magazine;

FIG. 11B is a top view illustrating selected aspects of the tissue anchor magazine of FIG. 11A;

FIG. 11C is a front view illustrating selected aspects of the tissue anchor magazine of FIG. 11A;

FIG. 12 schematically illustrates selected aspects of positioning and/or using the tissue anchor magazine of FIGS. 11A-11C in the operative space;

FIG. 13 schematically illustrates selected aspects of a tissue anchor magazine;

FIGS. 14-17 schematically illustrate selected aspects of positioning and/or using the tissue anchor magazine of FIG. 13 in the operative space;

FIG. 18 is a perspective view schematically illustrating selected aspects of a tissue anchor delivery device;

FIG. 19 is a cross-sectional view schematically illustrating selected aspects of the tissue anchor delivery device of FIG. 18;

FIGS. 20-23 schematically illustrate selected aspects related to using the tissue anchor delivery device of FIGS. 18-19;

FIGS. 24-25 schematically illustrate selected aspects of alternate configurations of the tissue anchor delivery device of FIGS. 18-19;

FIGS. 26-28 schematically illustrate selected aspects of alternate configurations of a tissue anchor advancement mechanism of the tissue anchor delivery device of FIGS. 18-19;

FIG. 29 is a perspective view schematically illustrating selected aspects of an alternative configuration of the tissue anchor delivery device of FIGS. 18-19;

FIG. 30 is a partial cutaway view schematically illustrating selected aspects of the tissue anchor delivery device of FIG. 29; and

FIGS. 31-33 schematically illustrate selected aspects related to using the tissue anchor delivery device of FIGS. 29-30.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. Like reference numerals indicate like elements throughout the views. The detailed description and drawings are intended to illustrate but not limit the disclosure. Those skilled in the art will recognize that the various elements described and/or shown may be arranged in various combinations and configurations without departing from the scope of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, enlightened by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

It is noted that references in the specification to “an embodiment”, “some embodiments”, “other embodiments”, etc., indicate that the embodiment(s) described may include one or more particular features, structures, and/or characteristics. However, such recitations do not necessarily mean that all embodiments include the particular features, structures, and/or characteristics. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, individual elements described herein, even if not explicitly shown in a particular combination, are contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The figures generally illustrate selected components and/or arrangements of medical devices, systems, and/or methods. It should be noted that in any given figure, some features may not be shown, or may be shown schematically, for simplicity. Additional details regarding some elements may be illustrated in other figures in greater detail. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to features or elements may be equally referred to all instances and quantities beyond one of said feature or element. As such, it will be understood that the following discussion may apply equally to any and/or all of the elements for which there are more than one within the medical devices, systems, and/or methods, unless explicitly stated to the contrary. Additionally, all instances of some elements or features may not be shown in each figure for clarity.

With its complexity, range of motion, and extensive use, a common soft tissue injury is damage to the rotator cuff or rotator cuff tendons. Damage to the rotator cuff is a potentially serious medical condition that may occur during hyperextension, from an acute traumatic tear, or from overuse of the joint. An accepted treatment for rotator cuff tears may include reattaching the torn tendon to the humeral head using sutures. Additionally, in treating rotator cuff tears, an accepted practice may also include the placement of a scaffold over the repaired tendon to mechanically reinforce the repaired tendon and/or to promote tissue reformation. Therefore, there is an ongoing need to deliver, position, and secure medical implants to soft tissue during an arthroscopic procedure in order to treat injuries to the rotator cuff, rotator cuff tendons, or other soft tissue or tendon injuries throughout a body. At least some of those procedures may involve the placement of multiple surgical anchors, and in some cases may involve multiple surgical anchor delivery devices. Repeatedly removing a surgical anchor delivery device to load a new surgical anchor onto the surgical anchor delivery device and then reinserting a surgical anchor delivery device to the treatment site to deliver surgical anchors thereto may extend a procedure, may create opportunity to drop a surgical anchor delivery device thereby causing the surgical anchor delivery device to become unsterile, may cause discomfort to the patient, and/or may cause or aggravate other risks. There is an ongoing need to deliver multiple surgical anchors within a single procedure without exchanging the surgical anchor delivery device and/or removing the surgical anchor delivery device between deployment of surgical anchors.

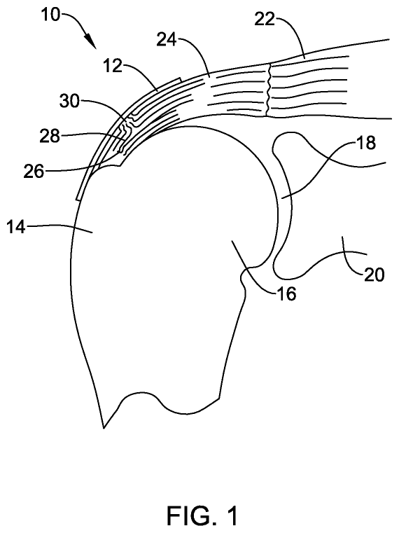

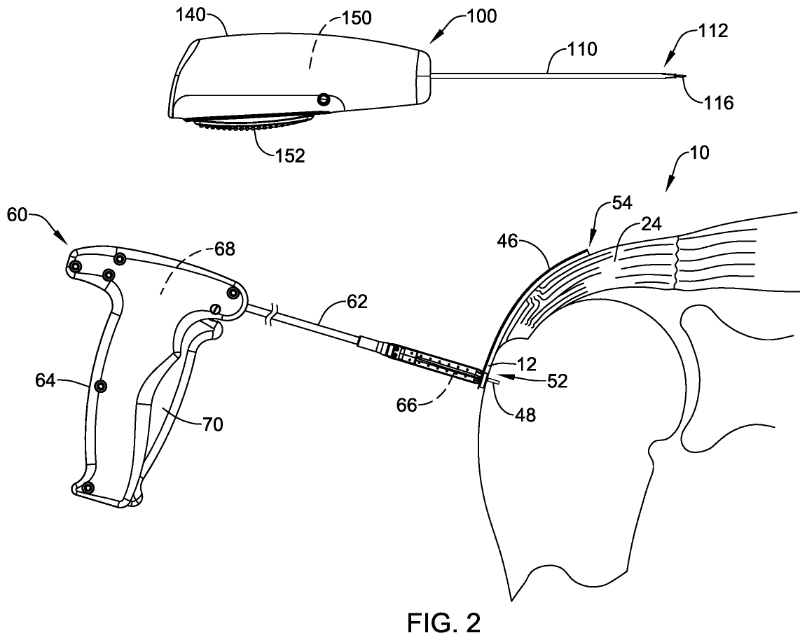

FIGS. 1 and 2 schematically illustrate selected aspects of a shoulder 10 including an implant 12 positioned at a treatment site within an operative space in a body of a patient. In at least some embodiments, the implant 12 may be a sheet-like implant and/or a scaffold configured to reinforce damaged tissue. In some embodiments, the implant 12 may include collagen and/or may be collagen based. FIGS. 1 and 2 further illustrate a head 14 of the humerus 16 mating with a glenoid fossa 18 of the scapula 20. The glenoid fossa 18 includes a shallow depression in the scapula 20. A supraspinatus tendon 22 is also shown. A distal tendon 24 of the supraspinatus tendon 22 meets the humerus 16 at an insertion point 26.

In FIGS. 1 and 2, the distal tendon 24 includes a damaged portion 28 located near the insertion point 26. The damaged portion 28 includes a tear 30 extending partially through the distal tendon 24. In some cases, the tear 30 may be a partial thickness tear. In some cases, the tear 30 may be a complete thickness tear. The tear 30 is depicted on the bursal side of the distal tendon 24, however, the tear 30 may also be on the opposite or articular side of the distal tendon 24 and/or may include internal tears to the distal tendon 24 not visible on either surface.

FIGS. 1 and 2 illustrate that the implant 12 has been placed over the tear 30. In this example, the implant 12 is placed on the bursal side of the distal tendon 24 regardless of whether the tear is on the bursal side, the articular side, or within the distal tendon 24. Further, the implant 12 may overlay multiple tears.

In some instances, delivery of the implant 12 to a treatment site of a patient may require a physician to create an incision in the patient (e.g., in the patient's skin, etc.) sufficient to access the treatment site. A system for delivering tissue anchors and/or the implant 12 into the operative space in the body of the patient may comprise an implant delivery device 60 including an elongate shaft 62 configured to deliver the implant 12 into the operative space. After creating the “access site,” the physician may deliver and/or insert the implant delivery device 60 through the incision and/or the access site into the operative space (e.g., subcutaneously and/or under the skin) and position a distal end of the implant delivery device 60 adjacent the treatment site and/or adjacent tissue (e.g., tendon, muscle, etc.) within the operative space. In some embodiments, the implant 12 may be advanced into the operative space within the elongate shaft 62 of the implant delivery device 60.

In some embodiments, the implant delivery device 60 may comprise a proximal handle 64 coupled to and/or disposed at a proximal end of the elongate shaft 62. In some embodiments, a support frame 46 may be coupled to an inner shaft 66 of the implant delivery device 60. In some embodiments, the support frame 46 may be detachably coupled to the inner shaft 66. The support frame 46 may be detachably coupled to the implant 12. In some embodiments, the support frame 46 and/or the implant 12 may be wrapped and/or folded upon itself within the elongate shaft 62 of the implant delivery device 60. In some embodiments, the support frame 46 and/or the implant 12 may be disposed distal of the inner shaft 66. The proximal handle 64 may include an actuation mechanism 68 configured to move and/or translate the elongate shaft 62 proximally relative to the inner shaft 66 and/or the proximal handle 64 to release and/or deploy the implant 12 and/or the support frame 46 detachably coupled to the implant 12. The implant delivery device 60 may comprise a trigger 70 operatively coupled to the actuation mechanism 68 such that actuation of the trigger 70 operates the actuation mechanism 68 to move and/or translate the elongate shaft 62 proximally relative to the inner shaft 66 and/or the proximal handle 64. The implant delivery device 60 may be manipulated to position and/or deploy the implant 12 at and/or adjacent the treatment site and/or adjacent tissue within the operative space.

When positioning and/or deploying the implant 12 at and/or adjacent the treatment site and/or adjacent tissue within the operative space, a user may orient the implant 12 such that a proximal portion 52 of the implant 12 may be adjacent (e.g., overlaid) on a portion of the humerus 16 (e.g., on the bone), while a distal portion 54 of the implant 12 may overlay the distal tendon 24, as seen in FIG. 2. In some embodiments, the support frame 46 may include a tack member 48 extending therefrom and/or extending through the implant 12. The tack member 48 may be configured to provide temporary fixation of the support frame 46 and/or the implant 12 to bone or tissue at the treatment site within the operative space.

In some embodiments, the elongate shaft 62 and/or the inner shaft 66 may be removable from the operative space after deploying the support frame 46 and/or the implant 12. In some embodiments, the support frame 46 and/or the implant 12 may be detached from the inner shaft 66 prior to removing the elongate shaft 62 and/or the inner shaft 66 from the operative space, thereby leaving the implant 12 and/or the support frame 46 disposed at and/or adjacent the treatment site and/or adjacent tissue within the operative space.

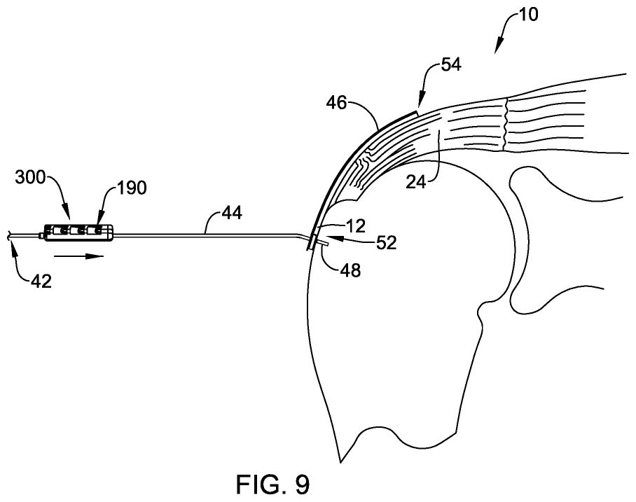

In some embodiments, the implant delivery device 60 may comprise a tether 44 (e.g., FIGS. 9-10, 12) extending proximally from the support frame 46 and/or the tack member 48. In some embodiments, the tether 44 may be coupled to the support frame 46 and/or the implant 12. In some embodiments, the tether 44 may be configured to extend to a free end 42 (e.g., FIGS. 9-10, 12) disposed outside of the operative space after the elongate shaft 62 of the implant delivery device 60 has been removed from the operative space. In some embodiments, the tether 44 may be configured to remove the support frame 46 and/or the tack member 48 from the operative space after the implant 12 has been secured and/or affixed to bone and/or tissue at the treatment site and/or within the operative space. While the tether 44 is expressly illustrated with respect to FIGS. 9-10 and 12, it shall be understood that the tether 44 may be used and/or present in other embodiments and/or configurations.

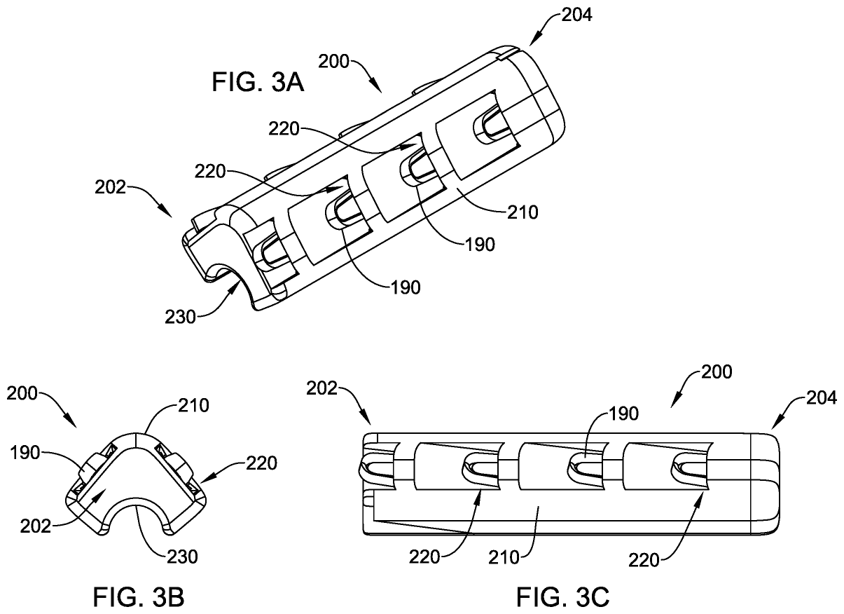

In at least some embodiments, the system may comprise a tissue anchor delivery device 100. The tissue anchor delivery device 100 may be configured to be advanced and/or inserted through the incision and/or the access site into the operative space. Once the implant 12 has been placed appropriately, it may be desirable to utilize the tissue anchor delivery device 100 to insert a tissue anchor (or multiple tissue anchors) of a plurality of tissue anchors 190 (e.g., FIGS. 3A-3C, 8A-8C, 11A-11C, 13), through the implant 12 into bone and/or tissue (e.g., the distal tendon 24 and/or the humerus 16) at the treatment site and/or within the operative space.

In some embodiments, an access sheath may be used during advancement and/or insertion of the tissue anchor delivery device 100 through the incision and/or the access site to the treatment site and/or into the operative space. The access sheath may cover a distal end region of the tissue anchor delivery device 100, thereby shielding portions of the shoulder 10 (e.g., tissue, muscle, etc.) from edges and/or sharp portions of the tissue anchor delivery device 100 as a distal end of the tissue anchor delivery device 100 is advanced to the treatment site and/or positioned adjacent the implant 12.

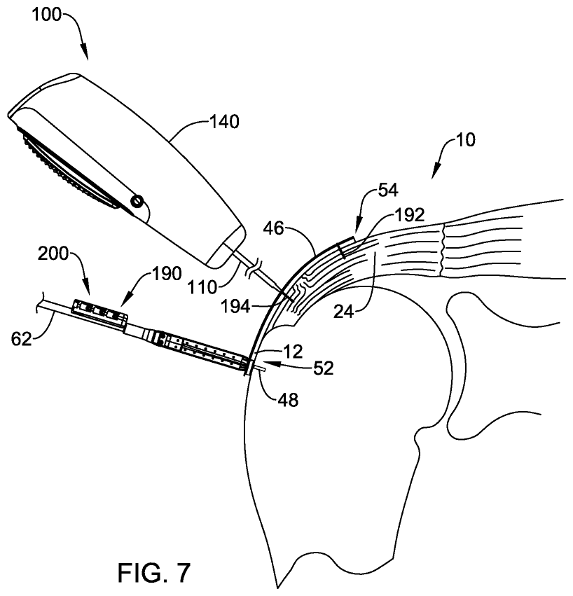

In at least some embodiments, once the tissue anchor delivery device 100 and the access sheath (where present) has been inserted through the incision and/or the access site and advanced to the treatment site and/or into the operative space, the access sheath may be removed. Removing the access sheath may expose portions of the tissue anchor delivery device 100 which may be utilized to affix the implant 12 to the humerus 16 and/or the distal tendon 24. For example, the implant 12 may be anchored to the humerus 16 using one or more bone anchors and/or the implant 12 may be anchored to the distal tendon 24 using a plurality of tissue anchors 190 arranged around a periphery of the implant 12 (e.g., FIG. 7).

In some embodiments, the tissue anchor delivery device 100 may comprise an elongate shaft 110 having a lumen extending therein to a distal end 112 of the elongate shaft 110. In some embodiments, the tissue anchor delivery device 100 may comprise a handle 140 disposed at a proximal end of the elongate shaft 110. In some embodiments, the handle 140 may include an actuation mechanism 150 configured to advance a tissue anchor out of the elongate shaft 110 and/or into the implant 12 and/or tissue at the treatment site and/or within the operative space.

In some embodiments, the elongate shaft 110 may extend distally away from the handle 140 to the distal end 112. In some embodiments, the handle 140 may include a handle housing. In some embodiments, the handle housing may comprise a first housing member and a second housing member designed to mate with one another, in a “clam shell” configuration, for example. Other configurations are also contemplated.

In some embodiments, the handle 140 may be coupled to the elongate shaft 110. In some embodiments, the elongate shaft 110 may be axially and/or rotationally secured relative to the handle 140. In some embodiments, the elongate shaft 110 may be fixedly attached to the handle 140. The elongate shaft 110 may include a wall defining an outer surface and the lumen extending therein. In some embodiments, the distal end 112 of the elongate shaft 110 may include a distal face. In some embodiments, the distal face may be oriented substantially perpendicular to a central longitudinal axis of the elongate shaft 110.

In some embodiments, the elongate shaft 110 may include at least one tine 116 extending distally away from the distal end 112 and/or the distal face of the elongate shaft 110. In at least some embodiments, the at least one tine 116 may extend parallel to the central longitudinal axis of the elongate shaft 110. In some embodiments, the at least one tine 116 may include exactly one tine, or the at least one tine 116 may include two tines, three tines, four tines, or more tines as needed or desired.

Collectively, the at least one tine 116 may define and/or border a passage through which a tissue anchor may pass through as the tissue anchor is advanced and/or deployed out of the elongate shaft 110. Further, the at least one tine 116 may be designed to create a pilot hole at the treatment site (e.g., within tissue and/or the implant 12). For example, after a user positions and/or aligns the distal end 112 of the elongate shaft 110 along the implant 12, the user may apply a force to the handle 140 and/or the elongate shaft 110 such that the at least one tine 116 pierces through the implant 12 and/or into tissue at the treatment site (e.g., the distal tendon 24), thereby creating a pilot hole for which a portion of a tissue anchor may be inserted.

In some embodiments, the at least one tine 116 may include at least one curved side (e.g., a concave surface facing a second opposed tine of the at least one tine 116) and a pointed end or tip. In some examples, a curved side of the at least one tine 116 may be configured to mate with and/or correspond to a portion of the tissue anchor in a complimentary manner. In some embodiments, the at least one tine 116 may take various shapes, such as spikes, spears, prongs, or other shapes. The at least one tine 116 may generally have a pointed distal end or tip for piercing tissue or bone.

In some embodiments, the tissue anchor delivery device 100 and/or the actuation mechanism 150 may comprise a trigger 152. In some embodiments, the trigger 152 may be and/or may include a lever, a knob, a button, a slider, etc. In some embodiments, the trigger 152 may be movable relative to the handle 140 and/or the handle housing to activate, operate, and/or actuate the actuation mechanism 150. In some embodiments, the trigger 152 may be secured to the handle 140 and/or the handle housing. In some embodiments, the trigger 152 may be secured to and/or pivotable relative to the handle 140 and/or the handle housing at a pivot point. Other configurations, while not expressly shown, are also contemplated. For example, in some embodiments, the trigger 152 may be secured to and/or slidable relative to the handle 140 and/or the handle housing. In some embodiments, the trigger 152 may be secured to and/or movable inward relative to the handle 140 and/or the handle housing (e.g., toward an interior of the handle 140 and/or the handle housing). In some embodiments, the trigger 152 may be a touch sensitive structure. Other configurations are also contemplated. It can be appreciated that the handle 140 may be designed such that a user may grasp the handle 140 and/or the handle housing with one hand and actuate and/or depress the trigger 152 with that same hand (e.g., via squeezing, etc.).

In some embodiments, the actuation mechanism 150 may be disposed within an interior of the tissue anchor delivery device 100, the handle 140, the handle housing, and/or the elongate shaft 110. In some embodiments, at least a portion of the trigger 152 may be disposed within the interior of the tissue anchor delivery device 100, the handle 140, and/or the handle housing.

In some previous tissue anchor systems, the delivery tool only holds and deploys a single tissue anchor at a time, thereby requiring the delivery tool to be removed from the operative space to load subsequent tissue anchors thereon before deploying them. Each exchange into and/or out of the operative space takes time and raises the risk of dropping and/or contaminating the delivery tool while permitting irrigation fluid from the operative space to be lost through the access site. At least some aspects of this disclosure address these factors by providing a system that does not require the tissue anchor delivery device 100 to be removed from the operative space to load subsequent tissue anchors thereon.

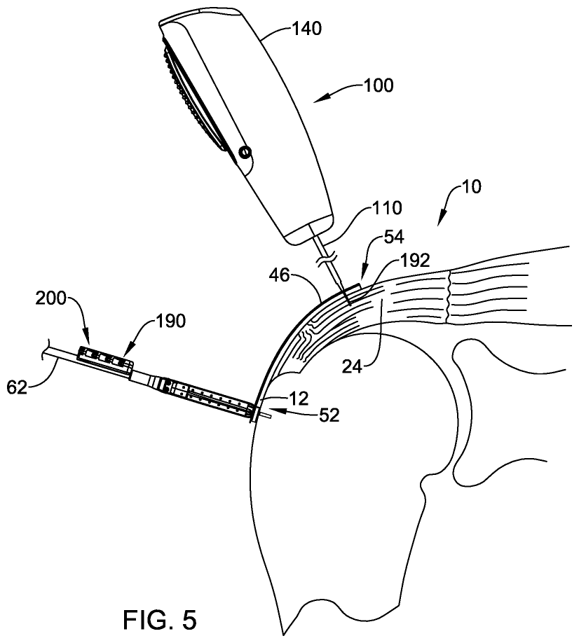

In some embodiments, the system may comprise a tissue anchor magazine 200, shown in FIGS. 3A-3C, separate and detached from the tissue anchor delivery device 100. The tissue anchor magazine 200 may have a plurality of tissue anchors 190 stored and/or disposed therein. The tissue anchor magazine 200 may be configured to be delivered subcutaneously into the operative space (e.g., under the skin) separate from the tissue anchor delivery device 100, as seen in FIG. 5 for example.

Returning to FIGS. 3A-3C, in some embodiments, the tissue anchor magazine 200 may comprise a body 210. In some embodiments, the body 210 may have a three-dimensional shape. In some embodiments, the body 210 may have a generally cuboid shape, a generally tetrahedron or polyhedron shape, etc. Other configurations are also contemplated. In some embodiments, the body 210 may be regularly shaped. In some embodiments, the body 210 may be irregularly shaped.

In some embodiments, the body 210 may include a plurality of recesses 220 extending at least partially into the body 210. Each recess of the plurality of recesses 220 may include and/or may hold one tissue anchor of the plurality of tissue anchors 190 therein. In some embodiments, the plurality of recesses 220 may comprise two recesses, three recesses, four recesses, five recesses, six recesses, seven recesses, eight recesses, etc. Other configurations are also contemplated.

In some embodiments, the tissue anchor magazine 200 may be multi-sided, with at least one recess of the plurality of recesses 220 formed in each side of the tissue anchor magazine 200. In some alternative configurations, the plurality of recesses 220 may be formed in a single side of the tissue anchor magazine 200. Other configurations are also contemplated.

In some embodiments, the plurality of recesses 220 may open towards a proximal end 202 of the tissue anchor magazine 200. In at least some embodiments, the plurality of recesses 220 may be formed at and/or may be oriented at an oblique angle to a longitudinal axis of the tissue anchor magazine 200. Accordingly, each tissue anchor of the plurality of tissue anchors 190 may be oriented at an oblique angle to the longitudinal axis of the tissue anchor magazine 200.

In some alternative configurations, the plurality of recesses 220 and/or each tissue anchor of the plurality of tissue anchors 190 may be oriented substantially perpendicular to the longitudinal axis of the tissue anchor magazine 200. In some further alternative configurations, the plurality of recesses 220 may open towards a distal end 204 of the tissue anchor magazine 200.

In some embodiments, engagement of the distal end 112 of the elongate shaft 110 of the tissue anchor delivery device 100 with the tissue anchor magazine 200 within the operative space and/or in situ may be configured to load one tissue anchor of the plurality of tissue anchors 190 onto the tissue anchor delivery device 100 in situ and/or within the operative space.

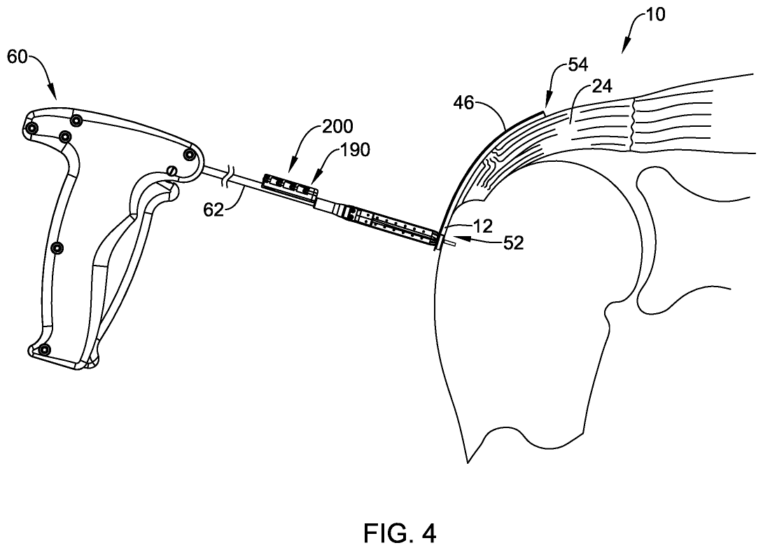

In some embodiments, the tissue anchor magazine 200 may be configured to be detachably coupled to a distal portion of the elongate shaft 62 of the implant delivery device 60, as seen in FIG. 4. Again returning to FIGS. 3A-3C, in at least some embodiments, the tissue anchor magazine 200 may comprise a curved outer surface 230 extending parallel to the longitudinal axis of the tissue anchor magazine 200. In some embodiments, the curved outer surface 230 may be concave and/or may extend into the body 210 of the tissue anchor magazine 200. The curved outer surface 230 may be configured to align with, receive, and/or engage with the elongate shaft 62 of the implant delivery device 60. In some embodiments, a radius of curvature of the curved outer surface 230 may be complementary to and/or may be substantially similar to a radius of curvature of an outer surface of the elongate shaft 62 of the implant delivery device 60. The curved outer surface 230 may be configured to mate with the elongate shaft 62 of the implant delivery device 60.

In some embodiments, the tissue anchor magazine 200 may be configured to releasably and/or detachably couple to the elongate shaft 62 of the implant delivery device 60. In some embodiments, the tissue anchor magazine 200 may be coupled to the elongate shaft 62 of the implant delivery device 60 via a snap fit, a friction fit, a releasable adhesive, a tongue and groove arrangement, a projection and slot arrangement, a hook and loop system, or other coupling structure.

In some embodiments, the tissue anchor magazine 200 may be replaceable (e.g., decoupled from the elongate shaft 62 of the implant delivery device 60, removed from the elongate shaft 62 of the implant delivery device 60, and subsequently replaced with a second tissue anchor magazine) during a procedure or treatment. In some embodiments, the tissue anchor magazine 200 may be replaceable within the operative space and/or in situ. In some embodiments, the elongate shaft 62 of the implant delivery device 60 may be at least partially removed from the operative space in order to remove and/or replace the tissue anchor magazine 200 and then reinserted into the operative space to continue the procedure or treatment after attaching the subsequent tissue anchor magazine to the elongate shaft 62 of the implant delivery device 60.

In some embodiments, a method of securing the implant 12 to tissue in the operative space in the body of the patient may comprise advancing the implant 12 into the operative space within the elongate shaft 62 of the implant delivery device 60. The method of securing the implant 12 to tissue in the operative space in the body of the patient may comprise deploying the implant 12 adjacent tissue (e.g., the distal tendon 24, etc.) within the operative space, as seen in FIG. 2.

In some embodiments, the method of securing the implant 12 to tissue in the operative space in the body of the patient may comprise positioning the tissue anchor magazine 200 within the operative space, the tissue anchor magazine 200 having the plurality of tissue anchors 190 stored therein, as seen in FIG. 4. In some embodiments, positioning the tissue anchor magazine 200 within the operative space may include attaching the tissue anchor magazine 200 to the distal portion of the elongate shaft 62 of the implant delivery device 60. In some embodiments, positioning the tissue anchor magazine 200 within the operative space may include attaching the tissue anchor magazine 200 to the distal portion of the elongate shaft 62 of the implant delivery device 60 before the elongate shaft 62 of the implant delivery device 60 is advanced into the operative space. In some alternative embodiments, positioning the tissue anchor magazine 200 within the operative space may include attaching the tissue anchor magazine 200 to the distal portion of the elongate shaft 62 of the implant delivery device 60 after the elongate shaft 62 of the implant delivery device 60 is advanced into the operative space.

In some embodiments, the method of securing the implant 12 to tissue in the operative space in the body of the patient may comprise advancing the elongate shaft 110 of the tissue anchor delivery device 100 into the operative space separately from the tissue anchor magazine 200, as seen in FIG. 5. In some embodiments, the method of securing the implant 12 to tissue in the operative space in the body of the patient may comprise deploying a first tissue anchor 192 (e.g., of the plurality of tissue anchors 190) from the elongate shaft 110 of the tissue anchor delivery device 100 into the implant 12, as shown in FIG. 5 for example.

In some embodiments, the first tissue anchor 192 of the plurality of tissue anchors 190 may be pre-loaded onto the tissue anchor delivery device 100 before advancing the elongate shaft 110 of the tissue anchor delivery device 100 into the operative space. In some embodiments, the elongate shaft 110 of the tissue anchor delivery device 100 may be advanced into the operative space with the first tissue anchor 192 loaded thereon. In such embodiments, the distal end 112 of the elongate shaft 110 of the tissue anchor delivery device 100 may be engaged with the tissue anchor magazine 200 and/or one tissue anchor of the plurality of tissue anchors 190 stored therein to load the first tissue anchor 192 of the plurality of tissue anchors 190 onto the tissue anchor delivery device 100. In such embodiments, the distal end 112 of the elongate shaft 110 of the tissue anchor delivery device 100 may be engaged with the tissue anchor magazine 200 and/or one tissue anchor of the plurality of tissue anchors 190 stored therein within the operative space to load the first tissue anchor 192 of the plurality of tissue anchors 190 onto the tissue anchor delivery device 100. In such embodiments, the distal end 112 of the elongate shaft 110 of the tissue anchor delivery device 100 may be engaged with the tissue anchor magazine 200 and/or one tissue anchor of the plurality of tissue anchors 190 stored therein outside of the operative space to load the first tissue anchor 192 of the plurality of tissue anchors 190 onto the tissue anchor delivery device 100.

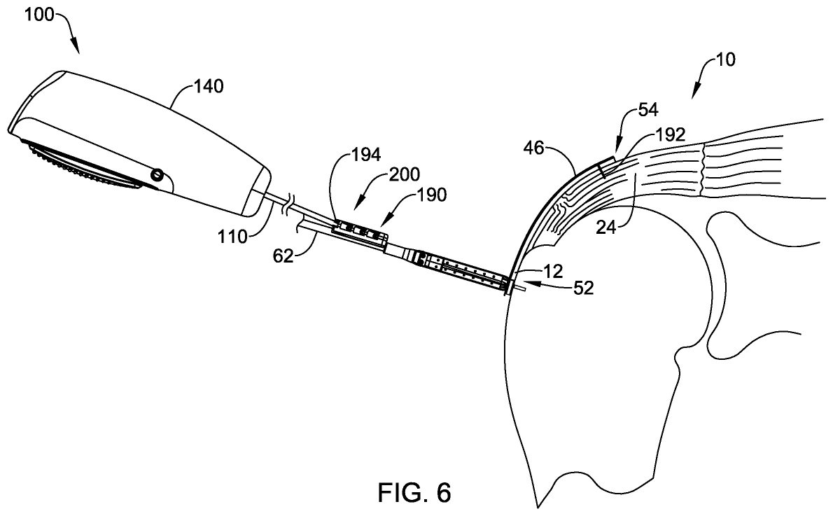

In some embodiments, after deploying the first tissue anchor 192 of the plurality of tissue anchors 190 from the elongate shaft 110 of the tissue anchor delivery device 100 into the implant 12, the method of securing the implant 12 to tissue in the operative space in the body of the patient may comprise thereafter, engaging the distal end 112 of the elongate shaft 110 of the tissue anchor delivery device 100 with the tissue anchor magazine 200 and/or the plurality of tissue anchors 190 stored therein to load a second tissue anchor 194 of the plurality of tissue anchors 190 from the plurality of tissue anchors 190 onto the tissue anchor delivery device 100 in situ and/or within the operative space, as seen in FIG. 6. By reloading the tissue anchor delivery device 100 in situ and/or within the operative space, procedure time may be shortened and/or risks associated with exchanging the tissue anchor delivery device 100 may be avoided.

In some embodiments, after loading the second tissue anchor 194 of the plurality of tissue anchors 190 from the plurality of tissue anchors 190 onto the tissue anchor delivery device 100, the method may comprise deploying the second tissue anchor 194 of the plurality of tissue anchors 190 from the elongate shaft 110 of the tissue anchor delivery device 100 into the implant 12, as shown in FIG. 7.

The steps and/or method illustrated in FIGS. 5-7 and described herein may be repeated as necessary to load and deliver additional tissue anchors into the implant 12 around a perimeter and/or a periphery of the implant 12 to secure the implant 12 to tissue (e.g., the distal tendon 24, etc.) within the operative space.

In some embodiments, the method may comprise removing the tissue anchor magazine 200 and/or the implant delivery device 60 from the operative space after securing the implant 12 to tissue. In some embodiments, removing the tissue anchor magazine 200 and/or the implant delivery device 60 from the operative space may include removing the support frame 46 and/or the tack member 48 from the operative space by retracting the implant delivery device 60 and/or the tether 44 (where present). As discussed herein, in some embodiments, the support frame 46 may be secured to the inner shaft 66 and/or the elongate shaft 62 of the implant delivery device 60. Accordingly, as the implant delivery device 60 is withdrawn and/or removed from the operative space, the support frame 46 and/or the tack member 48 will also be withdrawn from the operative space to leave the implant 12 in place adjacent tissue (e.g., the distal tendon 24, etc.) at the treatment site and/or within the operative space.

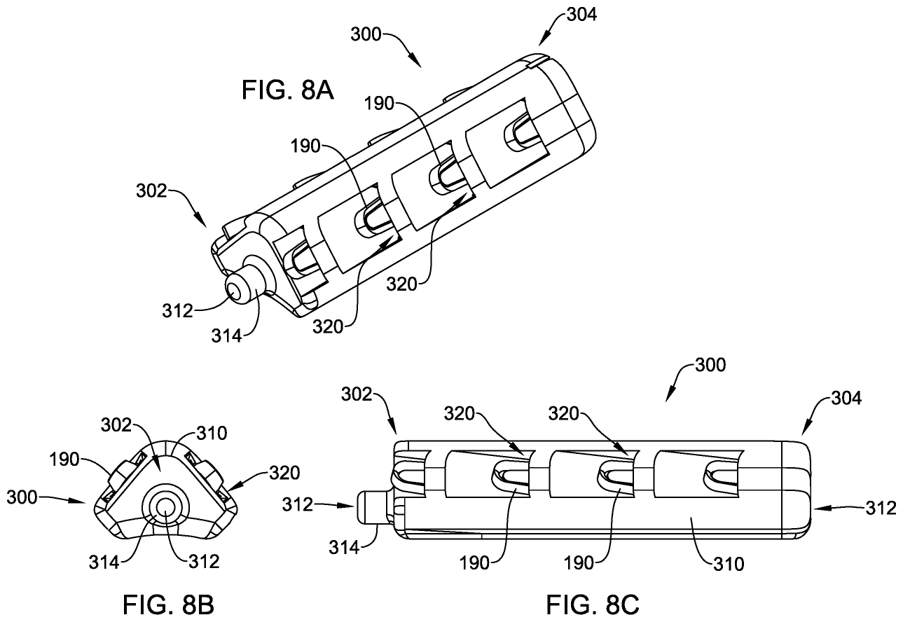

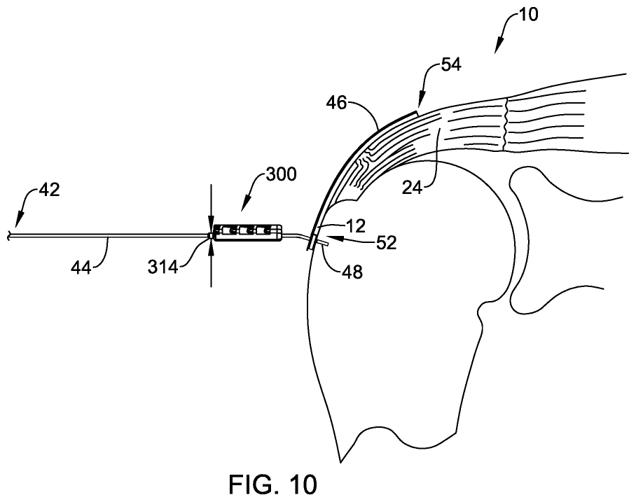

In some embodiments, the system may comprise a tissue anchor magazine 300, shown in FIGS. 8A-8C, separate and detached from the tissue anchor delivery device 100. The tissue anchor magazine 300 may have a plurality of tissue anchors 190 stored and/or disposed therein. The tissue anchor magazine 300 may be configured to be delivered subcutaneously into the operative space (e.g., under the skin) separate from the tissue anchor delivery device 100, as seen in FIGS. 9-10 for example.

Returning to FIGS. 8A-8C, in some embodiments, the tissue anchor magazine 300 may comprise a body 310. In some embodiments, the body 310 may have a three-dimensional shape. In some embodiments, the body 310 may have a generally cuboid shape, a generally tetrahedron or polyhedron shape, a generally cylindrical shape, etc. Other configurations are also contemplated. In some embodiments, the body 310 may be regularly shaped. In some embodiments, the body 310 may be irregularly shaped.

In some embodiments, the body 310 may include a plurality of recesses 320 extending at least partially into the body 310. Each recess of the plurality of recesses 320 may include and/or may hold one tissue anchor of the plurality of tissue anchors 190 therein. In some embodiments, the plurality of recesses 320 may comprise two recesses, three recesses, four recesses, five recesses, six recesses, seven recesses, eight recesses, etc. Other configurations are also contemplated.

In some embodiments, the tissue anchor magazine 300 may be multi-sided, with at least one recess of the plurality of recesses 320 formed in each side of the tissue anchor magazine 300. In some alternative configurations, the plurality of recesses 320 may be formed in a single side of the tissue anchor magazine 300. Other configurations are also contemplated.

In some embodiments, the plurality of recesses 320 may open towards a proximal end 302 of the tissue anchor magazine 300. In at least some embodiments, the plurality of recesses 320 may be formed at and/or may be oriented at an oblique angle to a longitudinal axis of the tissue anchor magazine 300. Accordingly, each tissue anchor of the plurality of tissue anchors 190 may be oriented at an oblique angle to the longitudinal axis of the tissue anchor magazine 300.

In some alternative configurations, the plurality of recesses 320 and/or each tissue anchor of the plurality of tissue anchors 190 may be oriented substantially perpendicular to the longitudinal axis of the tissue anchor magazine 300. In some further alternative configurations, the plurality of recesses 320 may open towards a distal end 304 of the tissue anchor magazine 300.

In some embodiments, engagement of the distal end 112 of the elongate shaft 110 of the tissue anchor delivery device 100 with the tissue anchor magazine 300 within the operative space and/or in situ may be configured to load one tissue anchor of the plurality of tissue anchors 190 onto the tissue anchor delivery device 100 in situ and/or within the operative space.

In some embodiments, the implant delivery device 60 may comprise a tether 44 (e.g., FIGS. 9-10) extending proximally from the support frame 46 and/or the tack member 48. In some embodiments, the tether 44 may be coupled to the support frame 46 and/or the implant 12. In some embodiments, the tether 44 may be configured to extend proximally to a free end 42 (e.g., FIGS. 9-10) disposed outside of the operative space after the elongate shaft 62 of the implant delivery device 60 has been removed from the operative space. In some embodiments, the tether 44 may be configured to remove the support frame 46 and/or the tack member 48 from the operative space after the implant 12 has been secured and/or affixed to bone and/or tissue at the treatment site and/or within the operative space.

In some embodiments, the tissue anchor magazine 300 and/or the body 310 of the tissue anchor magazine 300 may include a lumen 312 extending therethrough. In at least some embodiments, the lumen 312 may extend longitudinally and/or parallel to the longitudinal axis of the tissue anchor magazine 300 and/or the body 310. In some embodiments, the lumen 312 may extend completely through the tissue anchor magazine 300 and/or the body 310. In some embodiments, the lumen 312 may be configured to slidably receive the tether 44 therein such that the tissue anchor magazine 300 is advanceable over the tether 44 to the operative space after deployment of the implant 12 and/or after removal of the implant delivery device 60 and/or the elongate shaft 62 from the operative space, as seen in FIG. 9.

In some embodiments, the tissue anchor magazine 300 may comprise a proximal flange 314 extending proximally from the proximal end 302 of the tissue anchor magazine 300 and/or the body 310. The lumen 312 may extend through and/or within the proximal flange 314. In some configurations, the tissue anchor magazine 300 may comprise a distal flange extending distally from the distal end 304 of the tissue anchor magazine 300 and/or the body 310. The lumen 312 may extend through and/or within the distal flange.

In some embodiments, the tissue anchor magazine 300 may be axially securable to and/or relative to the tether 44 within the operative space. In some embodiments, the tissue anchor magazine 300 may be axially securable to and/or relative to the tether 44 via a cam mechanism or other coupling structure. In some embodiments, the tissue anchor magazine 300 may be axially securable to and/or relative to the tether 44 by crimping the proximal flange 314 and/or the distal flange onto and/or against the tether 44, as seen in FIG. 10.

In some embodiments, the tissue anchor magazine 300 may be replaceable (e.g., decoupled from the tether 44, removed from the tether 44, and subsequently replaced with a second tissue anchor magazine) during a procedure or treatment. In some embodiments, the tissue anchor magazine 300 may be replaceable within the operative space and/or in situ. In some embodiments, the tissue anchor magazine 300 may be replaceable without removing the tether 44 and/or the support frame 46 from the operative space.

In some embodiments, a method of securing the implant 12 to tissue in the operative space in the body of the patient may comprise advancing the implant 12 into the operative space within the elongate shaft 62 of the implant delivery device 60. The method of securing the implant 12 to tissue in the operative space in the body of the patient may comprise deploying the implant 12 adjacent tissue (e.g., the distal tendon 24, etc.) within the operative space, as seen in FIG. 2.

In some embodiments, the method of securing the implant 12 to tissue in the operative space in the body of the patient may comprise positioning the tissue anchor magazine 300 within the operative space, the tissue anchor magazine 300 having the plurality of tissue anchors 190 stored therein, as seen in FIGS. 9-10. In some embodiments, positioning the tissue anchor magazine 300 within the operative space may include sliding the tissue anchor magazine 300 over the tether 44 coupled to the implant 12 and/or the support frame 46 within the operative space, as seen in FIG. 9. In some embodiments, positioning the tissue anchor magazine 300 within the operative space may include securing the tissue anchor magazine 300 to and/or relative to the tether 44 within the operative space, as seen in FIG. 10. In some embodiments, positioning the tissue anchor magazine 300 within the operative space may include axially securing the tissue anchor magazine 300 to and/or relative to the tether 44 within the operative space.

In some embodiments, the method of securing the implant 12 to tissue in the operative space in the body of the patient may comprise advancing the elongate shaft 110 of the tissue anchor delivery device 100 into the operative space separately from the tissue anchor magazine 300, similar to the configuration seen in FIG. 5. In some embodiments, the method of securing the implant 12 to tissue in the operative space in the body of the patient may comprise deploying a first tissue anchor 192 (e.g., of the plurality of tissue anchors 190) from the elongate shaft 110 of the tissue anchor delivery device 100 into the implant 12, similarly to FIG. 5 for example.

In some embodiments, the first tissue anchor 192 of the plurality of tissue anchors 190 may be pre-loaded onto the tissue anchor delivery device 100 before advancing the elongate shaft 110 of the tissue anchor delivery device 100 into the operative space. In some embodiments, the elongate shaft 110 of the tissue anchor delivery device 100 may be advanced into the operative space with the first tissue anchor 192 loaded thereon. In such embodiments, the distal end 112 of the elongate shaft 110 of the tissue anchor delivery device 100 may be engaged with the tissue anchor magazine 300 and/or one tissue anchor of the plurality of tissue anchors 190 stored therein to load the first tissue anchor 192 of the plurality of tissue anchors 190 onto the tissue anchor delivery device 100. In such embodiments, the distal end 112 of the elongate shaft 110 of the tissue anchor delivery device 100 may be engaged with the tissue anchor magazine 300 and/or one tissue anchor of the plurality of tissue anchors 190 stored therein within the operative space to load the first tissue anchor 192 of the plurality of tissue anchors 190 onto the tissue anchor delivery device 100. In such embodiments, the distal end 112 of the elongate shaft 110 of the tissue anchor delivery device 100 may be engaged with the tissue anchor magazine 300 and/or one tissue anchor of the plurality of tissue anchors 190 stored therein outside of the operative space to load the first tissue anchor 192 of the plurality of tissue anchors 190 onto the tissue anchor delivery device 100.

In some embodiments, after deploying the first tissue anchor 192 of the plurality of tissue anchors 190 from the elongate shaft 110 of the tissue anchor delivery device 100 into the implant 12, the method of securing the implant 12 to tissue in the operative space in the body of the patient may comprise thereafter, engaging the distal end 112 of the elongate shaft 110 of the tissue anchor delivery device 100 with the tissue anchor magazine 300 and/or the plurality of tissue anchors 190 stored therein to load a second tissue anchor 194 of the plurality of tissue anchors 190 from the plurality of tissue anchors 190 onto the tissue anchor delivery device 100 in situ and/or within the operative space, similarly to FIG. 6 for example. By reloading the tissue anchor delivery device 100 in situ and/or within the operative space, procedure time may be shortened and/or risks associated with exchanging the tissue anchor delivery device 100 may be avoided.

In some embodiments, after loading the second tissue anchor 194 of the plurality of tissue anchors 190 from the plurality of tissue anchors 190 onto the tissue anchor delivery device 100, the method may comprise deploying the second tissue anchor 194 of the plurality of tissue anchors 190 from the elongate shaft 110 of the tissue anchor delivery device 100 into the implant 12, similarly to FIG. 7 for example.

The steps and/or method illustrated and/or described herein may be repeated as necessary to load and deliver additional tissue anchors into the implant 12 around a perimeter and/or a periphery of the implant 12 to secure the implant 12 to tissue (e.g., the distal tendon 24, etc.) within the operative space.

In some embodiments, the method may comprise removing the tissue anchor magazine 300 from the operative space after securing the implant 12 to tissue. In some embodiments, removing the tissue anchor magazine 300 from the operative space may include removing the support frame 46 and/or the tack member 48 from the operative space by retracting the tether 44. As discussed herein, in some embodiments, the support frame 46 may be secured to the tether 44. Accordingly, as the tether 44 is withdrawn and/or removed from the operative space, the support frame 46 and/or the tack member 48 will also be withdrawn from the operative space to leave the implant 12 in place adjacent tissue (e.g., the distal tendon 24, etc.) at the treatment site and/or within the operative space.

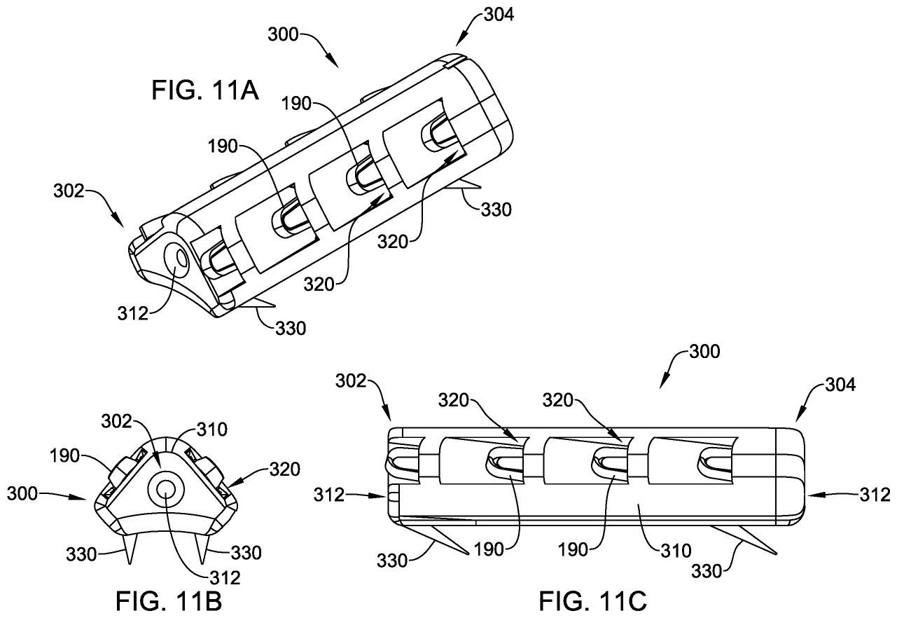

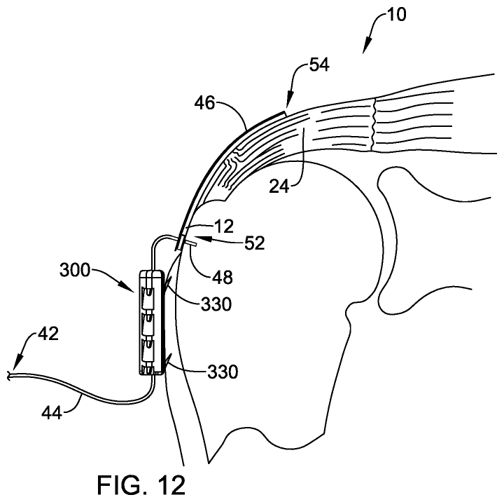

In some alternative embodiments, the system may comprise a tissue anchor magazine 300, shown in FIGS. 11A-11C, separate and detached from the tissue anchor delivery device 100. The tissue anchor magazine 300 may have a plurality of tissue anchors 190 stored and/or disposed therein, as discussed herein. The tissue anchor magazine 300 may be configured to be delivered subcutaneously into the operative space (e.g., under the skin) separate from the tissue anchor delivery device 100, as seen in FIG. 12 for example.

Returning to FIGS. 11A-11C, in some embodiments, the tissue anchor magazine 300 may comprise a body 310. In some embodiments, the body 310 may have a three-dimensional shape. In some embodiments, the body 310 may have a generally cuboid shape, a generally tetrahedron or polyhedron shape, a generally cylindrical shape, etc. Other configurations are also contemplated. In some embodiments, the body 310 may be regularly shaped. In some embodiments, the body 310 may be irregularly shaped.

In some embodiments, the body 310 may include a plurality of recesses 320 extending at least partially into the body 310. Each recess of the plurality of recesses 320 may include and/or may hold one tissue anchor of the plurality of tissue anchors 190 therein. In some embodiments, the plurality of recesses 320 may comprise two recesses, three recesses, four recesses, five recesses, six recesses, seven recesses, eight recesses, etc. Other configurations are also contemplated.

In some embodiments, the tissue anchor magazine 300 may be multi-sided, with at least one recess of the plurality of recesses 320 formed in each side of the tissue anchor magazine 300. In some alternative configurations, the plurality of recesses 320 may be formed in a single side of the tissue anchor magazine 300. Other configurations are also contemplated.

In some embodiments, the plurality of recesses 320 may open towards a proximal end 302 of the tissue anchor magazine 300. In at least some embodiments, the plurality of recesses 320 may be formed at and/or may be oriented at an oblique angle to a longitudinal axis of the tissue anchor magazine 300. Accordingly, each tissue anchor of the plurality of tissue anchors 190 may be oriented at an oblique angle to the longitudinal axis of the tissue anchor magazine 300.

In some alternative configurations, the plurality of recesses 320 and/or each tissue anchor of the plurality of tissue anchors 190 may be oriented substantially perpendicular to the longitudinal axis of the tissue anchor magazine 300. In some further alternative configurations, the plurality of recesses 320 may open towards a distal end 304 of the tissue anchor magazine 300.

In some embodiments, engagement of the distal end 112 of the elongate shaft 110 of the tissue anchor delivery device 100 with the tissue anchor magazine 300 within the operative space and/or in situ may be configured to load one tissue anchor of the plurality of tissue anchors 190 onto the tissue anchor delivery device 100 in situ and/or within the operative space.

In some embodiments, the implant delivery device 60 may comprise a tether 44 (e.g., FIG. 12) extending proximally from the support frame 46 and/or the tack member 48. In some embodiments, the tether 44 may be coupled to the support frame 46 and/or the implant 12. In some embodiments, the tether 44 may be configured to extend proximally to a free end 42 (e.g., FIG. 12) disposed outside of the operative space after the elongate shaft 62 of the implant delivery device 60 has been removed from the operative space. In some embodiments, the tether 44 may be configured to remove the support frame 46 and/or the tack member 48 from the operative space after the implant 12 has been secured and/or affixed to bone and/or tissue at the treatment site and/or within the operative space.

In some embodiments, the tissue anchor magazine 300 and/or the body 310 of the tissue anchor magazine 300 may include a lumen 312 extending therethrough. In at least some embodiments, the lumen 312 may extend longitudinally and/or parallel to the longitudinal axis of the tissue anchor magazine 300 and/or the body 310. In some embodiments, the lumen 312 may extend completely through the tissue anchor magazine 300 and/or the body 310. In some embodiments, the lumen 312 may be configured to slidably receive the tether 44 therein such that the tissue anchor magazine 300 is advanceable over the tether 44 to the operative space after deployment of the implant 12 and/or after removal of the implant delivery device 60 and/or the elongate shaft 62 from the operative space, as seen in FIG. 12.

In some embodiments, the tissue anchor magazine 300 may be axially securable relative to the tether 44 within the operative space. In some embodiments, the tissue anchor magazine 300 may be configured to be releasably coupled to tissue within the operative space. In some embodiments, the tissue anchor magazine 300 and/or the body 310 may comprise at least one anchoring barb 330 extending therefrom. The at least one anchoring barb 330 may be configured to releasably engage with tissue within the operative space to secure the tissue anchor magazine 300 to tissue within the operative space.

In some embodiments, the tissue anchor magazine 300 may be configured to be coupled to the distal tendon 24 near the implant 12. In some embodiments, the tissue anchor magazine 300 may be configured to be coupled to other tissue (e.g., muscle tissue, etc.) near the implant 12.

In some embodiments, the tissue anchor magazine 300 may be replaceable (e.g., decoupled from the tissue within the operative space, removed from the tether 44, and subsequently replaced with a second tissue anchor magazine) during a procedure or treatment. In some embodiments, the tissue anchor magazine 300 may be replaceable within the operative space and/or in situ. In some embodiments, the tissue anchor magazine 300 may be replaceable without removing the tether 44 and/or the support frame 46 from the operative space.

In some embodiments, a method of securing the implant 12 to tissue in the operative space in the body of the patient may comprise advancing the implant 12 into the operative space within the elongate shaft 62 of the implant delivery device 60. The method of securing the implant 12 to tissue in the operative space in the body of the patient may comprise deploying the implant 12 adjacent tissue (e.g., the distal tendon 24, etc.) within the operative space, as seen in FIG. 2.

In some embodiments, the method of securing the implant 12 to tissue in the operative space in the body of the patient may comprise positioning the tissue anchor magazine 300 within the operative space, the tissue anchor magazine 300 having the plurality of tissue anchors 190 (not shown) stored therein, as seen in FIG. 12. In some embodiments, positioning the tissue anchor magazine 300 within the operative space may include sliding the tissue anchor magazine 300 over the tether 44 coupled to the implant 12 and/or the support frame 46 within the operative space. In some embodiments, positioning the tissue anchor magazine 300 within the operative space may include axially securing the tissue anchor magazine 300 relative to the tether 44 and/or coupling the tissue anchor magazine 300 to tissue within the operative space.

In some embodiments, the method of securing the implant 12 to tissue in the operative space in the body of the patient may comprise advancing the elongate shaft 110 of the tissue anchor delivery device 100 into the operative space separately from the tissue anchor magazine 300, similar to the configuration seen in FIG. 5. In some embodiments, the method of securing the implant 12 to tissue in the operative space in the body of the patient may comprise deploying a first tissue anchor 192 (e.g., of the plurality of tissue anchors 190) from the elongate shaft 110 of the tissue anchor delivery device 100 into the implant 12, similarly to FIG. 5 for example.

In some embodiments, the first tissue anchor 192 of the plurality of tissue anchors 190 may be pre-loaded onto the tissue anchor delivery device 100 before advancing the elongate shaft 110 of the tissue anchor delivery device 100 into the operative space. In some embodiments, the elongate shaft 110 of the tissue anchor delivery device 100 may be advanced into the operative space with the first tissue anchor 192 loaded thereon. In such embodiments, the distal end 112 of the elongate shaft 110 of the tissue anchor delivery device 100 may be engaged with the tissue anchor magazine 300 and/or one tissue anchor of the plurality of tissue anchors 190 stored therein to load the first tissue anchor 192 of the plurality of tissue anchors 190 onto the tissue anchor delivery device 100. In such embodiments, the distal end 112 of the elongate shaft 110 of the tissue anchor delivery device 100 may be engaged with the tissue anchor magazine 300 and/or one tissue anchor of the plurality of tissue anchors 190 stored therein within the operative space to load the first tissue anchor 192 of the plurality of tissue anchors 190 onto the tissue anchor delivery device 100. In such embodiments, the distal end 112 of the elongate shaft 110 of the tissue anchor delivery device 100 may be engaged with the tissue anchor magazine 300 and/or one tissue anchor of the plurality of tissue anchors 190 stored therein outside of the operative space to load the first tissue anchor 192 of the plurality of tissue anchors 190 onto the tissue anchor delivery device 100.

In some embodiments, after deploying the first tissue anchor 192 of the plurality of tissue anchors 190 from the elongate shaft 110 of the tissue anchor delivery device 100 into the implant 12, the method of securing the implant 12 to tissue in the operative space in the body of the patient may comprise thereafter, engaging the distal end 112 of the elongate shaft 110 of the tissue anchor delivery device 100 with the tissue anchor magazine 300 and/or the plurality of tissue anchors 190 stored therein to load a second tissue anchor 194 of the plurality of tissue anchors 190 from the plurality of tissue anchors 190 onto the tissue anchor delivery device 100 in situ and/or within the operative space, similarly to FIG. 6 for example. By reloading the tissue anchor delivery device 100 in situ and/or within the operative space, procedure time may be shortened and/or risks associated with exchanging the tissue anchor delivery device 100 may be avoided.

In some embodiments, after loading the second tissue anchor 194 of the plurality of tissue anchors 190 from the plurality of tissue anchors 190 onto the tissue anchor delivery device 100, the method may comprise deploying the second tissue anchor 194 of the plurality of tissue anchors 190 from the elongate shaft 110 of the tissue anchor delivery device 100 into the implant 12, similarly to FIG. 7 for example.

The steps and/or method illustrated and/or described herein may be repeated as necessary to load and deliver additional tissue anchors into the implant 12 around a perimeter and/or a periphery of the implant 12 to secure the implant 12 to tissue (e.g., the distal tendon 24, etc.) within the operative space.

In some embodiments, the method may comprise removing the tissue anchor magazine 300 from the operative space after securing the implant 12 to tissue. In some embodiments, removing the tissue anchor magazine 300 from the operative space may include removing the support frame 46 and/or the tack member 48 from the operative space by retracting the tether 44. As discussed herein, in some embodiments, the support frame 46 may be secured to the tether 44. Accordingly, as the tether 44 is withdrawn and/or removed from the operative space, the support frame 46 and/or the tack member 48 will also be withdrawn from the operative space to leave the implant 12 in place adjacent tissue (e.g., the distal tendon 24, etc.) at the treatment site and/or within the operative space.

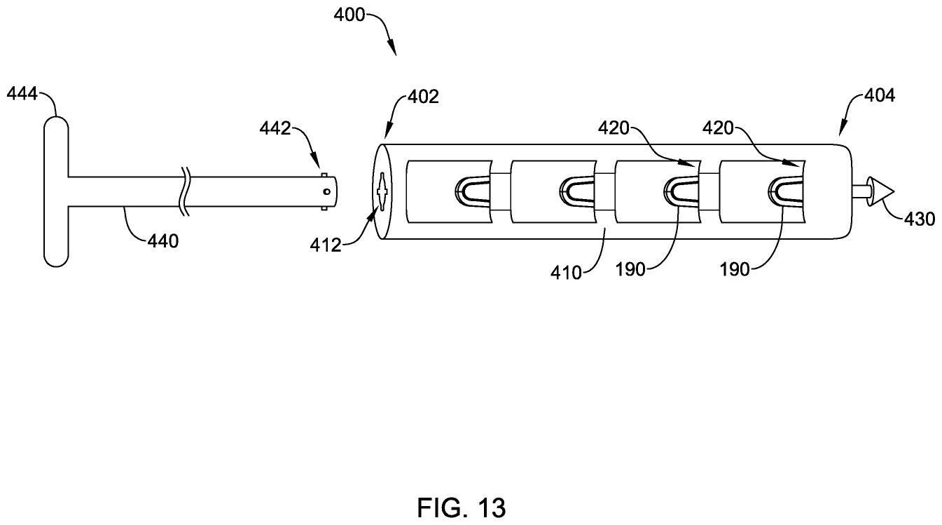

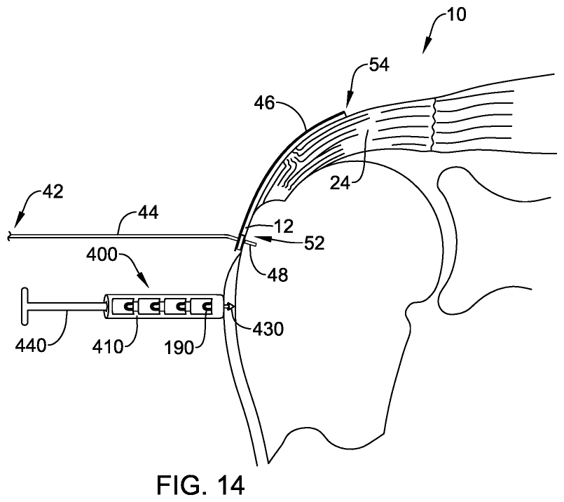

In some embodiments, the system may comprise a tissue anchor magazine 400, shown in FIG. 13, separate and detached from the tissue anchor delivery device 100. The tissue anchor magazine 400 may have a plurality of tissue anchors 190 stored and/or disposed therein, as discussed herein. The tissue anchor magazine 400 may be configured to be delivered subcutaneously into the operative space (e.g., under the skin) separate from the tissue anchor delivery device 100, as seen in FIGS. 14-16 for example.

Returning to FIG. 13, in some embodiments, the tissue anchor magazine 400 may comprise a body 410. In some embodiments, the body 410 may have a three-dimensional shape. In some embodiments, the body 410 may have a generally cuboid shape, a generally tetrahedron or polyhedron shape, a generally cylindrical shape, etc. Other configurations are also contemplated. In some embodiments, the body 410 may be regularly shaped. In some embodiments, the body 410 may be irregularly shaped.

In some embodiments, the body 410 may include a plurality of recesses 420 extending at least partially into the body 410. Each recess of the plurality of recesses 420 may include and/or may hold one tissue anchor of the plurality of tissue anchors 190 therein. In some embodiments, the plurality of recesses 420 may comprise two recesses, three recesses, four recesses, five recesses, six recesses, seven recesses, eight recesses, etc. Other configurations are also contemplated.

In some embodiments, the tissue anchor magazine 400 may be multi-sided, with at least one recess of the plurality of recesses 420 formed in each side of the tissue anchor magazine 400. In some alternative configurations, the plurality of recesses 420 may be formed in a single side of the tissue anchor magazine 400. Other configurations are also contemplated.

In some embodiments, the plurality of recesses 420 may open towards a proximal end 402 of the tissue anchor magazine 400. In at least some embodiments, the plurality of recesses 420 may be formed at and/or may be oriented at an oblique angle to a longitudinal axis of the tissue anchor magazine 400. Accordingly, each tissue anchor of the plurality of tissue anchors 190 may be oriented at an oblique angle to the longitudinal axis of the tissue anchor magazine 400.

In some alternative configurations, the plurality of recesses 420 and/or each tissue anchor of the plurality of tissue anchors 190 may be oriented substantially perpendicular to the longitudinal axis of the tissue anchor magazine 400. In some further alternative configurations, the plurality of recesses 420 may open towards a distal end 404 of the tissue anchor magazine 400.

In some embodiments, engagement of the distal end 112 of the elongate shaft 110 of the tissue anchor delivery device 100 with the tissue anchor magazine 400 within the operative space and/or in situ may be configured to load one tissue anchor of the plurality of tissue anchors 190 onto the tissue anchor delivery device 100 in situ and/or within the operative space.

In some embodiments, the implant delivery device 60 may comprise a tether 44 (e.g., FIGS. 14-16) extending proximally from the support frame 46 and/or the tack member 48. In some embodiments, the tether 44 may be coupled to the support frame 46 and/or the implant 12. In some embodiments, the tether 44 may be configured to extend proximally to a free end 42 (e.g., FIGS. 14-17) disposed outside of the operative space after the elongate shaft 62 of the implant delivery device 60 has been removed from the operative space. In some embodiments, the tether 44 may be configured to remove the support frame 46 and/or the tack member 48 from the operative space after the implant 12 has been secured and/or affixed to bone and/or tissue at the treatment site and/or within the operative space.

In some embodiments, the tissue anchor magazine 400 and/or the body 410 may comprise at least one anchoring barb 430 extending therefrom, as seen in FIG. 13. In some embodiments, the tissue anchor magazine 400 may be configured to be releasably coupled to tissue within the operative space (e.g., FIGS. 14-17). In some embodiments, the tissue anchor magazine 400 may be configured to be coupled to the distal tendon 24 near the implant 12. In some embodiments, the tissue anchor magazine 400 may be configured to be coupled to other tissue (e.g., muscle tissue, etc.) near the implant 12.

The at least one anchoring barb 430 may be configured to releasably engage with tissue within the operative space to secure the tissue anchor magazine 400 to tissue within the operative space. In some embodiments, the at least one anchoring barb 430 may be disposed at and/or may extend distally from the distal end 404 of the tissue anchor magazine 400 and/or the body 410.

In some embodiments, the tissue anchor magazine 400 may be configured to releasably couple with a delivery handle 440 configured to insert the tissue anchor magazine 400 into the operative space. In some embodiments, the proximal end 402 of the tissue anchor magazine 400 and/or the body 410 may include an aperture 412 formed therein. In some embodiments, a distal end 442 of the delivery handle 440 may be configured to engage with and/or be received by the aperture 412. In some embodiments, the delivery handle 440 may comprise a T-handle 444 at a proximal end thereof, but this is not required. Other handle shapes and/or features (e.g., a knob, a grip, etc.) may also be included or used. The delivery handle 440 may be at least partially rotatable with respect to the body 410 to lock and/or unlock the delivery handle 440 axially with respect to the body 410. In some embodiments, the aperture 412 may include a slot or groove formed therein that is configured to receive a projection extending outward from the delivery handle 440.

In some embodiments, the at least one anchoring barb 430 may be axially extendable from and/or retractable into the body 410. In some embodiments, actuation and/or manipulation of the delivery handle 440 relative to the body 410 may be configured to extend and/or retract the at least one anchoring barb 430 with respect to the body 410. In some embodiments, actuation and/or manipulation of the delivery handle 440 relative to the body 410 to extend and/or retract the at least one anchoring barb 430 with respect to the body 410 may include applying axial force (e.g., proximally and/or distally) to the delivery handle 440 while the delivery handle 440 is engaged with the body 410. In some embodiments, actuation and/or manipulation of the delivery handle 440 relative to the body 410 to extend and/or retract the at least one anchoring barb 430 with respect to the body 410 may include applying rotational force to the delivery handle 440 while the delivery handle 440 is engaged with the body 410. In some embodiments, actuation and/or manipulation of the delivery handle 440 relative to the body 410 to extend and/or retract the at least one anchoring barb 430 with respect to the body 410 may include applying axial and rotational force to the delivery handle 440 while the delivery handle 440 is engaged with the body 410.

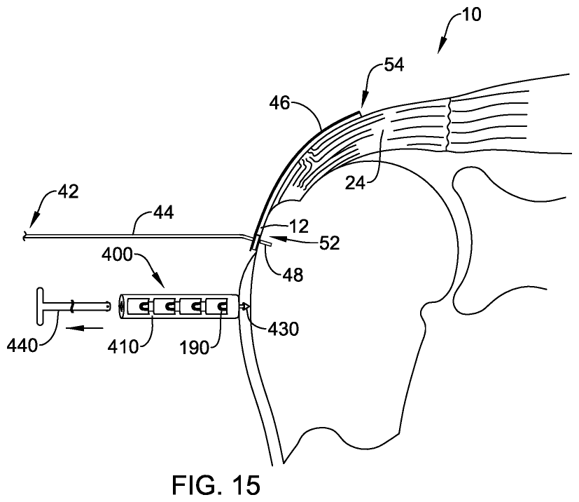

In some embodiments, the delivery handle 440 may be detachable from the body 410 to leave the body 410 and the plurality of tissue anchors 190 disposed within the operative space as the implant 12 is secured to tissue (e.g., the distal tendon 24, etc.), as seen in FIG. 15. In some embodiments, rotational and/or axial force may be applied to the delivery handle 440 to disengage the delivery handle 440 from the body 410. The delivery handle 440 may be removed from the body 410 as the plurality of tissue anchors 190 is deployed to free up space within the operative space and/or within the access site.

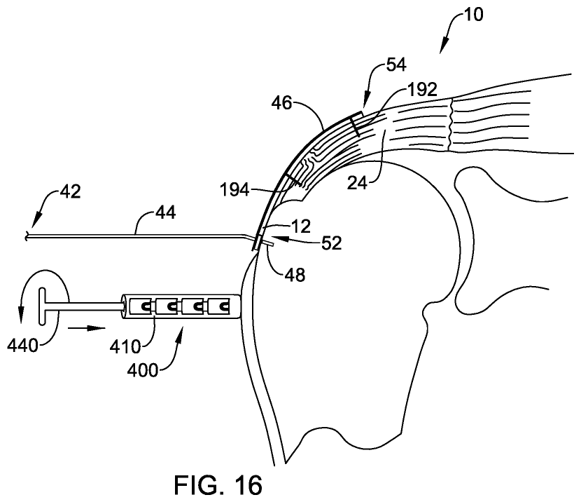

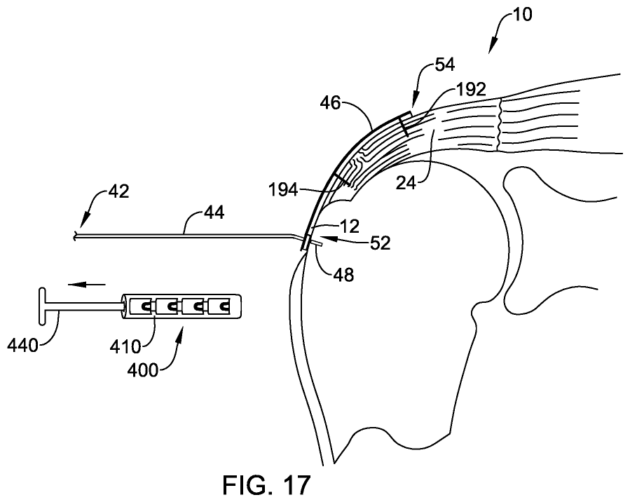

After deployment of the plurality of tissue anchors 190 (e.g., the first tissue anchor 192, the second tissue anchor 194, etc.) into the implant 12 and/or tissue, the delivery handle 440 may be re-engaged with the body 410 to remove the body 410 from the operative space. In some embodiments, as seen in FIG. 16 for example, the delivery handle 440 may be actuated and/or manipulated relative to the body 410 to retract the at least one anchoring barb 430 into the body 410, thereby decoupling the tissue anchor magazine 400 and/or the body 410 from tissue within the operative space. Thereafter, the tissue anchor magazine 400 and/or the body 410 may be withdrawn from the operative space using the delivery handle 440, as seen in FIG. 17. In some embodiments, the tissue anchor magazine 400 and/or the body 410 may be withdrawn from the operative space without retracting the at least one anchoring barb 430 into the body 410. In such embodiments, the delivery handle 440 may be pulled proximally to disengage the at least one anchoring barb 430 from tissue within the operative space, thereby decoupling the tissue anchor magazine 400 and/or the body 410 from tissue within the operative space.

In some embodiments, the tissue anchor magazine 400 may be replaceable (e.g., decoupled from the tissue within the operative space, removed from the operative space, and subsequently replaced with a second tissue anchor magazine) during a procedure or treatment. In some embodiments, the tissue anchor magazine 400 may be replaceable within the operative space and/or in situ. In some embodiments, the tissue anchor magazine 400 may be replaceable without removing the tether 44 and/or the support frame 46 from the operative space.

In some embodiments, a method of securing the implant 12 to tissue in the operative space in the body of the patient may comprise advancing the implant 12 into the operative space within the elongate shaft 62 of the implant delivery device 60. The method of securing the implant 12 to tissue in the operative space in the body of the patient may comprise deploying the implant 12 adjacent tissue (e.g., the distal tendon 24, etc.) within the operative space, as seen in FIG. 2.

In some embodiments, the method of securing the implant 12 to tissue in the operative space in the body of the patient may comprise positioning the tissue anchor magazine 400 within the operative space, the tissue anchor magazine 400 having the plurality of tissue anchors 190 stored therein, as seen in FIG. 14. In some embodiments, positioning the tissue anchor magazine 400 within the operative space may include axially securing the tissue anchor magazine 400 to tissue within the operative space.

In some embodiments, the method of securing the implant 12 to tissue in the operative space in the body of the patient may comprise detaching and/or decoupling the delivery handle 440 from the body 410 of the tissue anchor magazine 400. In some embodiments, the method of securing the implant 12 to tissue in the operative space in the body of the patient may comprise removing the delivery handle 440 from the operative space while leaving the body 410 of the tissue anchor magazine 400 secured to tissue within the operative space, as seen in FIG. 15.

In some embodiments, the method of securing the implant 12 to tissue in the operative space in the body of the patient may comprise advancing the elongate shaft 110 of the tissue anchor delivery device 100 into the operative space separately from the tissue anchor magazine 400, similar to the configuration seen in FIG. 5. In some embodiments, the method of securing the implant 12 to tissue in the operative space in the body of the patient may comprise deploying a first tissue anchor 192 (e.g., of the plurality of tissue anchors 190) from the elongate shaft 110 of the tissue anchor delivery device 100 into the implant 12, similarly to FIG. 5 for example.

In some embodiments, the first tissue anchor 192 of the plurality of tissue anchors 190 may be pre-loaded onto the tissue anchor delivery device 100 before advancing the elongate shaft 110 of the tissue anchor delivery device 100 into the operative space. In some embodiments, the elongate shaft 110 of the tissue anchor delivery device 100 may be advanced into the operative space with the first tissue anchor 192 loaded thereon. In such embodiments, the distal end 112 of the elongate shaft 110 of the tissue anchor delivery device 100 may be engaged with the tissue anchor magazine 400 and/or one tissue anchor of the plurality of tissue anchors 190 stored therein to load the first tissue anchor 192 of the plurality of tissue anchors 190 onto the tissue anchor delivery device 100. In such embodiments, the distal end 112 of the elongate shaft 110 of the tissue anchor delivery device 100 may be engaged with the tissue anchor magazine 400 and/or one tissue anchor of the plurality of tissue anchors 190 stored therein within the operative space to load the first tissue anchor 192 of the plurality of tissue anchors 190 onto the tissue anchor delivery device 100. In such embodiments, the distal end 112 of the elongate shaft 110 of the tissue anchor delivery device 100 may be engaged with the tissue anchor magazine 400 and/or one tissue anchor of the plurality of tissue anchors 190 stored therein outside of the operative space to load the first tissue anchor 192 of the plurality of tissue anchors 190 onto the tissue anchor delivery device 100.

In some embodiments, after deploying the first tissue anchor 192 of the plurality of tissue anchors 190 from the elongate shaft 110 of the tissue anchor delivery device 100 into the implant 12, the method of securing the implant 12 to tissue in the operative space in the body of the patient may comprise thereafter, engaging the distal end 112 of the elongate shaft 110 of the tissue anchor delivery device 100 with the tissue anchor magazine 400 and/or the plurality of tissue anchors 190 stored therein to load a second tissue anchor 194 of the plurality of tissue anchors 190 from the plurality of tissue anchors 190 onto the tissue anchor delivery device 100 in situ and/or within the operative space, similarly to FIG. 6 for example. By reloading the tissue anchor delivery device 100 in situ and/or within the operative space, procedure time may be shortened and/or risks associated with exchanging the tissue anchor delivery device 100 may be avoided.

In some embodiments, after loading the second tissue anchor 194 of the plurality of tissue anchors 190 from the plurality of tissue anchors 190 onto the tissue anchor delivery device 100, the method may comprise deploying the second tissue anchor 194 of the plurality of tissue anchors 190 from the elongate shaft 110 of the tissue anchor delivery device 100 into the implant 12, similarly to FIG. 7 for example.

The steps and/or method illustrated and/or described herein may be repeated as necessary to load and deliver additional tissue anchors into the implant 12 around a perimeter and/or a periphery of the implant 12 to secure the implant 12 to tissue (e.g., the distal tendon 24, etc.) within the operative space.

In some embodiments, the method may comprise, after deploying the plurality of tissue anchors 190 (e.g., the first tissue anchor 192, the second tissue anchor 194, etc.), recoupling the delivery handle 440 to the body 410 within the operative space, as seen in FIG. 16. In at least some embodiments, the method may comprise, after recoupling the delivery handle 440 to the body 410 within the operative space, retracting the at least one anchoring barb 430 into the body 410, thereby decoupling the tissue anchor magazine 400 and/or the body 410 from tissue within the operative space. In some embodiments, retracting the at least one anchoring barb 430 into the body 410 may comprise actuating and/or manipulating the delivery handle 440 relative to the body 410. In some embodiments, actuating and/or manipulating the delivery handle 440 relative to the body 410 may comprise applying axial and/or rotational force to the delivery handle 440.

In some embodiments, the method may comprise removing the tissue anchor magazine 400 from the operative space after securing the implant 12 to tissue, as seen in FIG. 17. In some embodiments, the method may further comprise removing the support frame 46 and/or the tack member 48 from the operative space by retracting the tether 44. As discussed herein, in some embodiments, the support frame 46 may be secured to the tether 44. Accordingly, as the tether 44 is withdrawn and/or removed from the operative space, the support frame 46 and/or the tack member 48 will also be withdrawn from the operative space to leave the implant 12 in place adjacent tissue (e.g., the distal tendon 24, etc.) at the treatment site and/or within the operative space.

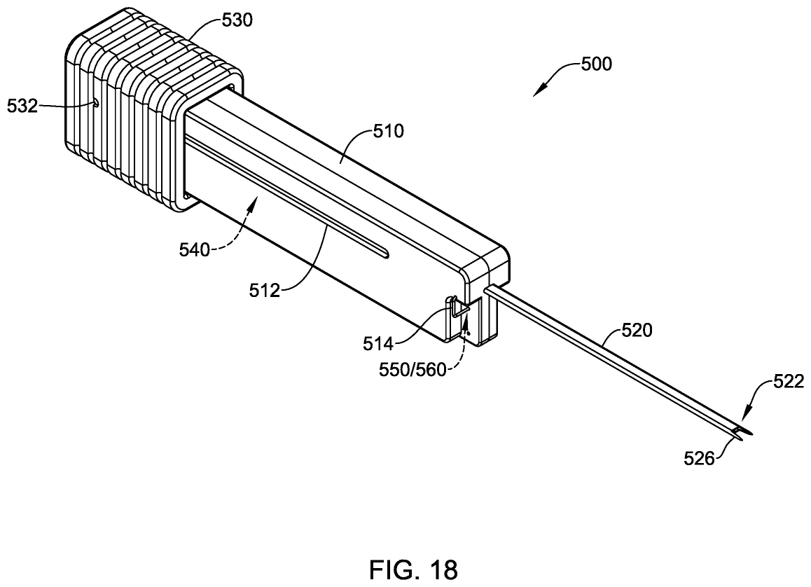

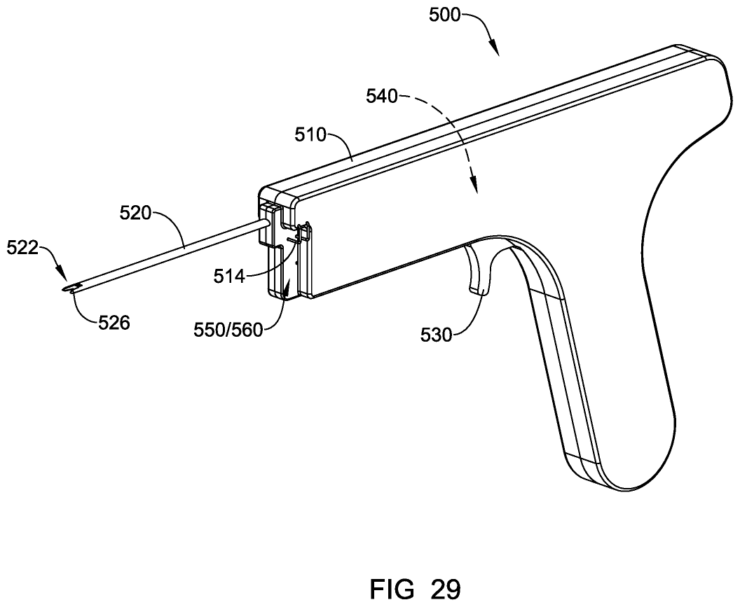

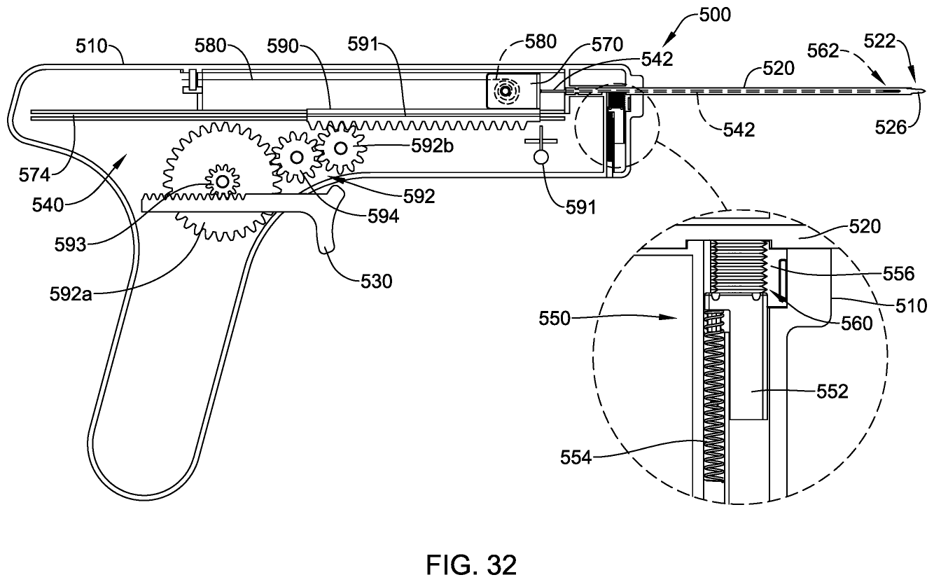

FIGS. 18-33 illustrate selected aspects of configurations for a tissue anchor delivery device 500. In some embodiments, the tissue anchor delivery device 500 may be used in place of the tissue anchor delivery device 100 described herein. In some embodiments, the tissue anchor delivery device 500 may comprise a handle 510 and an elongate shaft 520 extending distally from the handle 510. In some embodiments, the handle 510 may include a handle housing. In some embodiments, the handle housing may comprise a first housing member and a second housing member designed to mate with one another, in a “clam shell” configuration, for example. Other configurations are also contemplated.

In some embodiments, the handle 510 may be coupled to the elongate shaft 520. In some embodiments, the elongate shaft 520 may be axially and/or rotationally secured relative to the handle 510. In some embodiments, the elongate shaft 520 may be fixedly attached to the handle 510. The elongate shaft 520 may include a wall defining an outer surface and the lumen extending therein.

The elongate shaft 520 may have a length from the handle 510 to a distal end 522 of the elongate shaft 520 of at least 6 centimeters (cm). In some embodiments, the elongate shaft 520 may have a length from the handle 510 to a distal end 522 of the elongate shaft 520 of at least 8 centimeters (cm). In some embodiments, the elongate shaft 520 may have a length from the handle 510 to a distal end 522 of the elongate shaft 520 of at least 10 centimeters (cm). In some embodiments, the elongate shaft 520 may have a length from the handle 510 to a distal end 522 of the elongate shaft 520 of at least 12 centimeters (cm). In some embodiments, the elongate shaft 520 may have a length from the handle 510 to a distal end 522 of the elongate shaft 520 of at least 14 centimeters (cm). Other configurations are also contemplated.

In some embodiments, the elongate shaft 520 may include at least one tine 526 extending distally away from the distal end 522 of the elongate shaft 520. In at least some embodiments, the at least one tine 526 may extend parallel to the central longitudinal axis of the elongate shaft 520. In some embodiments, the at least one tine 526 may include exactly one tine, or the at least one tine 526 may include two tines, three tines, four tines, or more tines as needed or desired.

Collectively, the at least one tine 526 may define and/or border a passage through which a tissue anchor may pass through as the tissue anchor is advanced and/or deployed out of the elongate shaft 520. Further, the at least one tine 526 may be designed to create a pilot hole at the treatment site (e.g., within tissue and/or the implant 12). For example, after a user positions and/or aligns the distal end 522 of the elongate shaft 520 along the implant 12, the user may apply a force to the handle 510 and/or the elongate shaft 520 such that the at least one tine 526 pierces through the implant 12 and/or into tissue at the treatment site (e.g., the distal tendon 24), thereby creating a pilot hole for which a portion of a tissue anchor may be inserted.

In some embodiments, the at least one tine 526 may include at least one curved side (e.g., a concave surface facing a second opposed tine of the at least one tine 526) and a pointed end or tip. In some examples, a curved side of the at least one tine 526 may be configured to mate with and/or correspond to a portion of the tissue anchor in a complimentary manner. In some embodiments, the at least one tine 526 may take various shapes, such as spikes, spears, prongs, or other shapes. The at least one tine 526 may generally have a pointed distal end or tip for piercing tissue or bone.

In some embodiments, the tissue anchor delivery device 500 may comprise a trigger 530 movably coupled to the handle 510. The trigger 530 may be configured to actuate a tissue anchor advancement mechanism 540 disposed within the handle 510 and/or the elongate shaft 520.

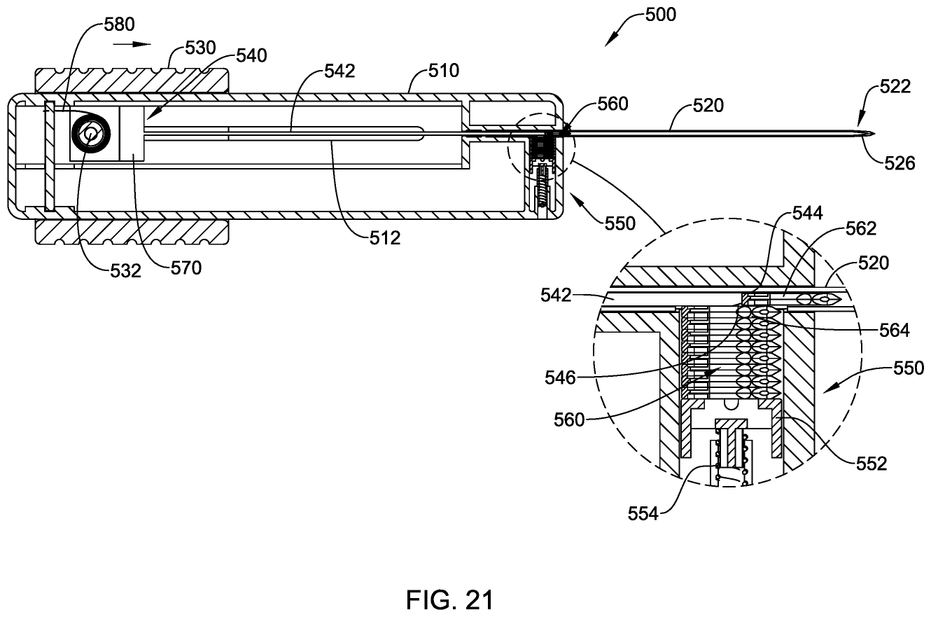

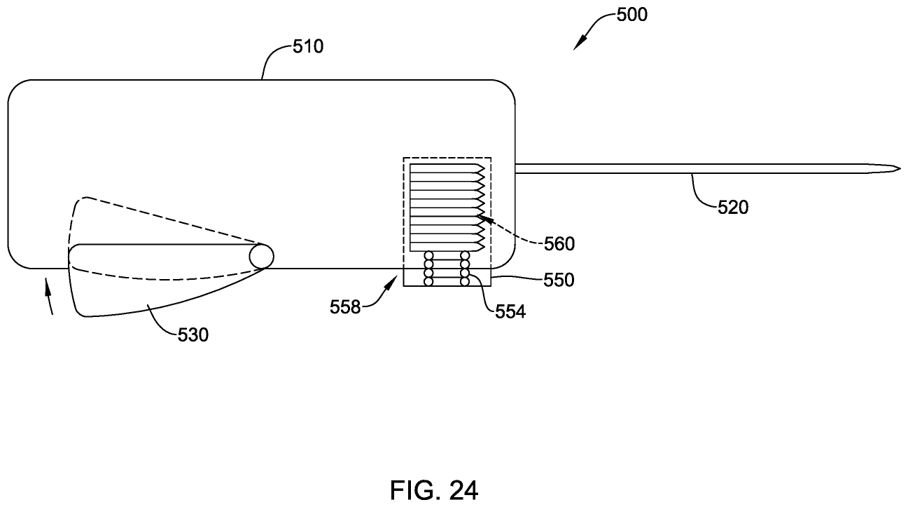

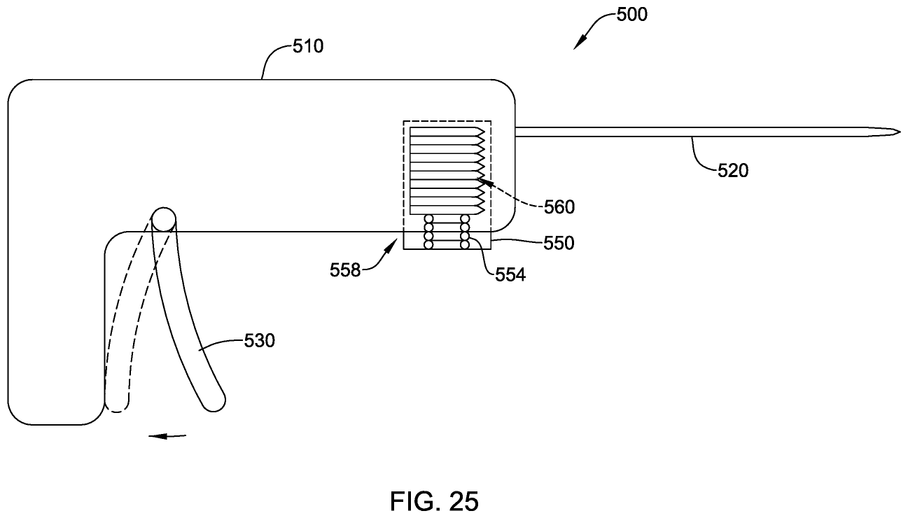

In some embodiments, the trigger 530 may be and/or may include a lever, a knob, a button, a slider, etc. In some embodiments, the trigger 530 may be movable relative to the handle 510 and/or the handle housing to activate, operate, and/or actuate the tissue anchor advancement mechanism 540. In some embodiments, the trigger 530 may be secured to the handle 510 and/or the handle housing. In some embodiments, the trigger 530 may be slidably coupled to the handle 510 and/or the handle housing, as seen in FIGS. 21-23 and 29-33. In some embodiments, the trigger 530 may be pivotably coupled to the handle 510 and/or the handle housing at a pivot point, as seen in FIGS. 24-25. In some embodiments, the trigger 530 may be secured to and/or movable inward relative to the handle 510 and/or the handle housing (e.g., toward an interior of the handle 510 and/or the handle housing). In some embodiments, the trigger 530 may be a touch sensitive structure. Other configurations are also contemplated. In some embodiments, the handle 510 may be designed such that a user may grasp the handle 510 and/or the handle housing with one hand and actuate the trigger 530 with that same hand (e.g., via squeezing, etc.). In some embodiments, the handle 510 may be designed such that a user may grasp the handle 510 and/or the handle housing with one hand and actuate the trigger 530 with another hand (e.g., via sliding, etc.). Other configurations are also contemplated.

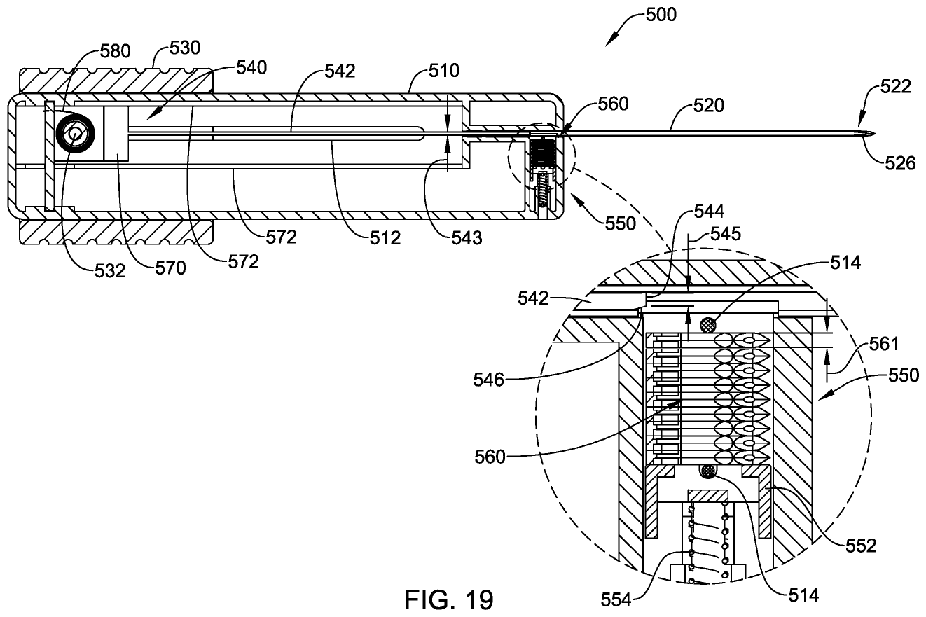

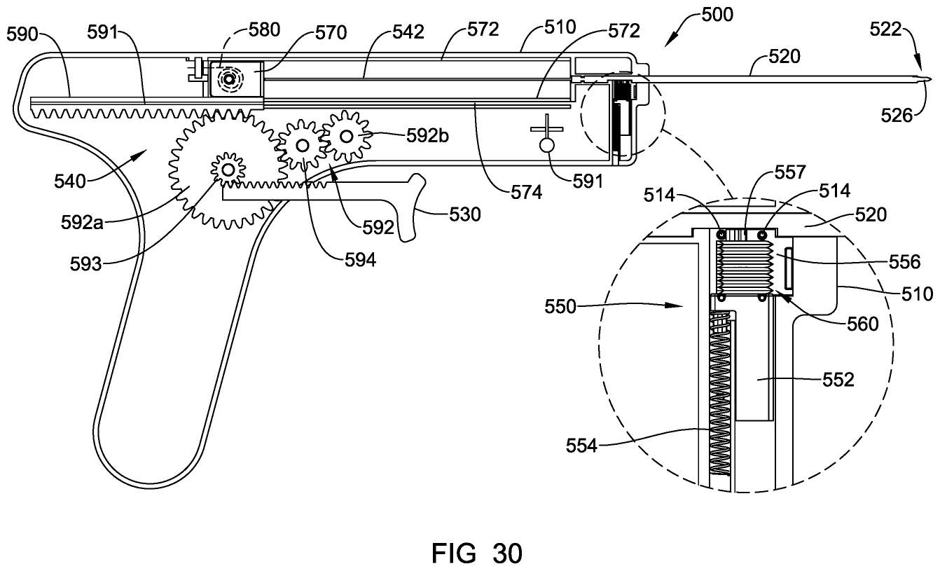

In some embodiments, the tissue anchor delivery device 500 may comprise a tissue anchor advancement mechanism 540 disposed within the handle 510 and/or the elongate shaft 520, as seen in FIGS. 19 and 30. In some embodiments, the tissue anchor advancement mechanism 540 may be disposed within an interior of the tissue anchor delivery device 500, the handle 510, the handle housing, and/or the elongate shaft 520. In some embodiments, at least a portion of the trigger 530 may be disposed within the interior of the tissue anchor delivery device 500, the handle 510, and/or the handle housing. The trigger 530 may be operatively coupled to the tissue anchor advancement mechanism 540. In one example, the handle 510 may include a longitudinally oriented slot 512. In some embodiments, a pin 532 or other engagement structure coupled to the trigger 530 may extend through the longitudinally oriented slot 512 and/or at least a portion of the tissue anchor advancement mechanism 540 such that movement of the trigger 530 relative to the handle 510 and/or the handle housing causes actuation and/or movement of the tissue anchor advancement mechanism 540 relative to the handle 510, the handle housing, and/or the elongate shaft 520. In some embodiments, the handle 510 and/or the handle housing may comprise at least one guide configured to receive the trigger 530 and/or configured to permit sliding movement of the trigger 530 therein.

In some embodiments, the tissue anchor delivery device 500 may comprise a tissue anchor magazine 550 coupled to the handle 510 of the tissue anchor delivery device 500 in communication with the elongate shaft 520. The tissue anchor magazine 550 may have a plurality of tissue anchors 560 disposed therein. In some embodiments, the tissue anchor magazine 550 may be integrally formed with the handle 510, as seen in FIGS. 18-23 and 29-33. In some embodiments, the tissue anchor magazine 550 may be configured to be removably received within a receptacle formed in the handle 510, as seen in FIGS. 24-25. For example, the tissue anchor magazine 550 may be a separate structure that may be inserted into and/or removed from the handle 510, such as to reload the tissue anchor delivery device 500 with additional tissue anchors. Other configurations are also contemplated, and various features of the illustrated examples may be combined and/or interchanged with each other (e.g., the integrated tissue anchor magazine of FIGS. 18-23 and 29-33 may be used with the handle of FIGS. 24-25, the removable tissue anchor magazine of FIGS. 24-25 may be used with the handle of FIGS. 18-23 and 29-33, etc.).

In some embodiments, the plurality of tissue anchors 560 may be stacked immediately adjacent to one another within the tissue anchor magazine 550. In some embodiments, the plurality of tissue anchors 560 may be stacked within the tissue anchor magazine 550 such that adjacent tissue anchors of the plurality of tissue anchors 560 are in direct contact with each other. In some embodiments, the tissue anchor magazine 550 may be configured to guide and/or direct the plurality of tissue anchors 560 toward and/or into the elongate shaft 520.

In some embodiments, the tissue anchor magazine 550 may comprise a pushing element 552 (e.g., FIGS. 19-21 and 30-33) configured to push and/or translate the plurality of tissue anchors 560 toward and/or into the elongate shaft 520. The tissue anchor magazine 550 may comprise a biasing element 554 (e.g., FIGS. 19-21 and 30-33), such as a spring (e.g., a coil spring, etc.), configured to continuously bias and/or urge the pushing clement 552 and/or the plurality of tissue anchors 560 toward the elongate shaft 520. The biasing element 554 may be disposed opposite the plurality of tissue anchors 560 with respect to the pushing element 552 (e.g., the pushing element 552 may be disposed between the biasing element 554 and the plurality of tissue anchors 560).

In some embodiments, the tissue anchor delivery device 500 may comprise a locking pin 514 configured to removably engage with the handle 510 and the plurality of tissue anchors 560, as seen in FIGS. 18-19 and 29-30, to prevent movement and/or translation of the plurality of tissue anchors 560 toward and/or into the elongate shaft 520. In some embodiments, the tissue anchor delivery device 500 may comprise a plurality of locking pins (ref. 514) configured to removably engage with the handle 510 and the plurality of tissue anchors 560. In some embodiments, the locking pin 514 and/or the plurality of locking pins (ref. 514) may be configured to engage with the tissue anchor magazine 550 and/or the pushing clement 552 to prevent movement and/or translation of the pushing clement 552 and/or the plurality of tissue anchors 560 toward the elongate shaft 520. In some embodiments, the tissue anchor delivery device 500 may be supplied with the locking pin 514 and/or the plurality of locking pins (ref. 514) in place to prevent accidental actuation and/or deployment of the plurality of tissue anchors 560. In some embodiments, the tissue anchor delivery device 500 may be supplied with the locking pin 514 and/or the plurality of locking pins (ref. 514) in place to permit and/or maintain sterilization of the plurality of tissue anchors 560. In some embodiments, the locking pin 514 and/or the plurality of locking pins (ref. 514) may be a U-shaped structure configured to engage with the handle 510 and multiple elements and/or structures within the handle 510 (e.g., the plurality of tissue anchors 560 and the pushing element 552, etc.) of the tissue anchor delivery device 500. In some embodiments, the locking pin 514 and/or the plurality of locking pins (ref. 514) may be a straight structure configured to engage with the handle 510 and a single element or structure within the handle 510 (e.g., only the plurality of tissue anchors 560, only the pushing element 552, etc.) of the tissue anchor delivery device 500. Other configurations are also contemplated.

In some embodiments, the tissue anchor advancement mechanism 540 may be configured to advance the plurality of tissue anchors 560 from the tissue anchor magazine 550 out the distal end 522 of the elongate shaft 520. In some embodiments, the tissue anchor advancement mechanism 540 may be configured to advance only one tissue anchor of the plurality of tissue anchors 560 along the length of the elongate shaft 520 at a time. In some embodiments, each actuation of the tissue anchor advancement mechanism 540 may advance the only one tissue anchor of the plurality of tissue anchors 560 along an entirety of the length of the elongate shaft 520 (e.g., from the tissue anchor magazine 550 to and/or out the distal end 522 of the elongate shaft 520). In some embodiments, each actuation of the tissue anchor advancement mechanism 540 may advance the only one tissue anchor of the plurality of tissue anchors 560 only a portion of the length of the elongate shaft 520.

In some embodiments, the tissue anchor advancement mechanism 540 may comprise a pusher 542 configured to engage the only one tissue anchor of the plurality of tissue anchors 560. Additional details related to the pusher 542 are shown in FIGS. 19-21 and may be included in any other example and/or configuration disclosed herein, even if not explicitly shown. In at least some embodiments, the pusher 542 may comprise a distal face 544 configured to abut at least a portion of the only one tissue anchor of the plurality of tissue anchors 560. The pusher 542 may comprise a bevel 546 extending proximally from the distal face 544 to a bottom surface 548 of the pusher 542 such that a height 545 of the distal face 544 is less than a thickness 543 of the pusher 542 and the height 545 of the distal face 544 is equal to or less than a thickness 561 of the only one tissue anchor of the plurality of tissue anchors 560.

In some embodiments, the tissue anchor delivery device 500 and/or the tissue anchor advancement mechanism 540 may comprise a slide block 570 coupled to the pusher 542, as seen in FIGS. 19-23 and 30-33. In some embodiments, the slide block 570 may be fixedly attached to a proximal end of the pusher 542. Other configurations are also contemplated. In some embodiments, the slide block 570 may be operatively coupled to the trigger 530. In some embodiments, the handle 510 may comprise a longitudinally oriented guide 572 disposed within the handle 510, the longitudinally oriented guide 572 being configured to receive the slide block 570 therein. In some embodiments, the pin 532 or other engagement structure may be coupled to the slide block 570. In some embodiments, the pin 532 or other engagement structure may be fixedly attached to the slide block 570. In some embodiments, the pin 532 or other engagement structure may be integrally formed with the slide block 570 and extend outward therefrom. Other configurations are also contemplated.

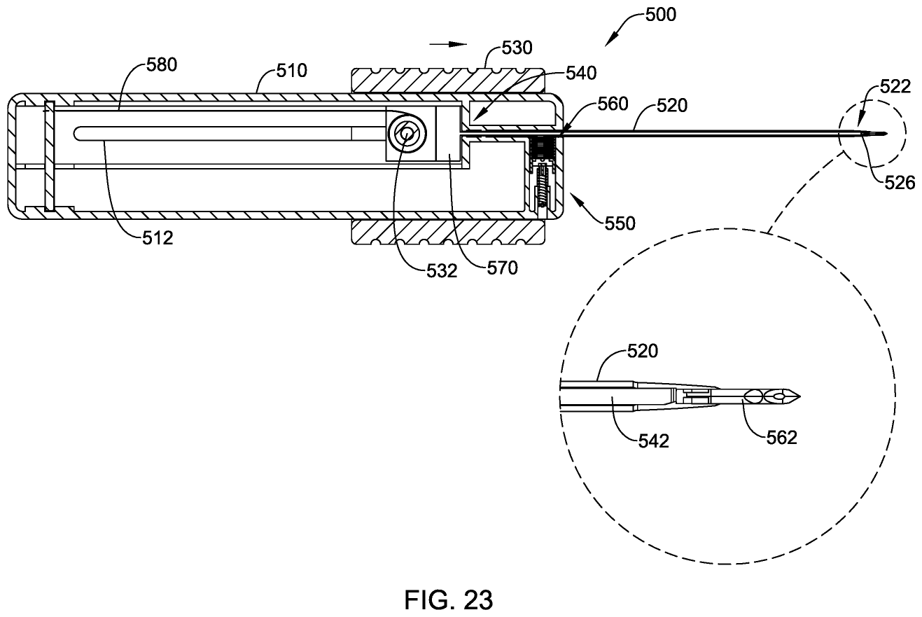

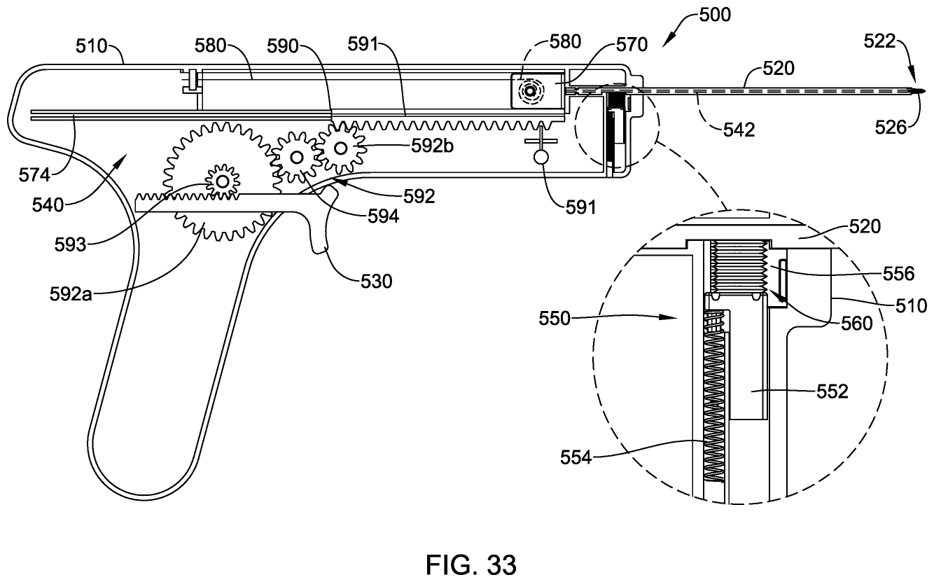

The slide block 570 may be movable longitudinally within the handle 510 between a proximal position (e.g., FIGS. 19-20 and 29-31) and a distal position (e.g., FIGS. 23 and 33) in response to actuation of the trigger 530 and/or the tissue anchor advancement mechanism 540. In the proximal position, the distal face 544 of the pusher 542 may be disposed proximal of the plurality of tissue anchors 560. In the distal position, the distal face 544 of the pusher 542 may be disposed at and/or proximate the distal end 522 of the elongate shaft 520.

In some embodiments, the tissue anchor delivery device 500 and/or the tissue anchor advancement mechanism 540 may comprise an automatic retraction apparatus 580 configured to reset the tissue anchor advancement mechanism 540 and/or return the slide block 570 to the proximal position. In some embodiments, the automatic retraction apparatus 580 may be a constant force spring. In some embodiments, the automatic retraction apparatus 580 may be coupled to the slide block 570. In some embodiments, the automatic retraction apparatus 580 may be coiled and/or wrapped around the pin 532 or other engagement structure. In some embodiments, the automatic retraction apparatus 580 may be fixedly attached to a portion of the handle 510. In some embodiments, the automatic retraction apparatus 580 may be a compression spring. In some embodiments, the automatic retraction apparatus 580 may be disposed around (e.g., may encircle) the pusher 542 and/or may be disposed between the slide block 570 and the elongate shaft 520 and/or a distal end or a distal portion of the handle 510. Other configurations are also contemplated. In some embodiments, the slide block 570 may be biased toward a proximal end of the handle 510 and/or the proximal position by the automatic retraction apparatus 580. In some embodiments, the slide block 570 may be continuously biased toward the proximal end of the handle 510 and/or the proximal position by the automatic retraction apparatus 580.

A method of securing an implant 12 to tissue in an operative space in a body of a patient may comprise advancing the implant 12 into the operative space within the elongate shaft 62 of the implant delivery device 60. The method of securing the implant 12 to tissue in the operative space in the body of the patient may comprise deploying the implant 12 adjacent tissue (e.g., the distal tendon 24, etc.) within the operative space, similarly to FIG. 2 for example.

The method of securing the implant 12 to tissue in the operative space in the body of the patient may comprise inserting the tissue anchor delivery device 500 into the operative space, similarly to FIG. 5 for example. The distal end 522 of the elongate shaft 520 may be disposed proximate and/or against the implant 12. In some embodiments, the method of securing the implant 12 to tissue in the operative space in the body of the patient may comprise urging and/or inserting the at least one tine 526 into the implant 12 and/or tissue (e.g., the distal tendon 24, etc.) within the operative space.

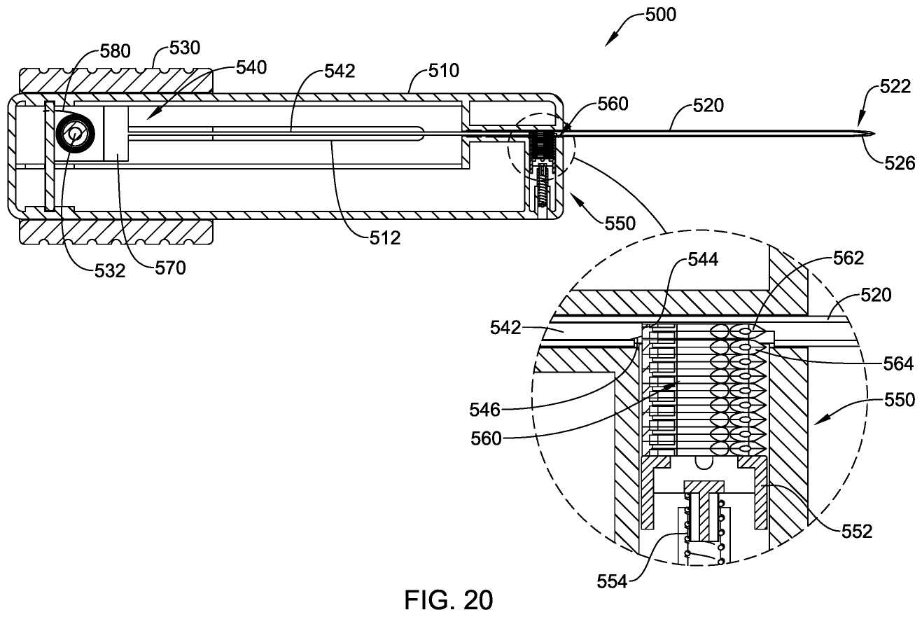

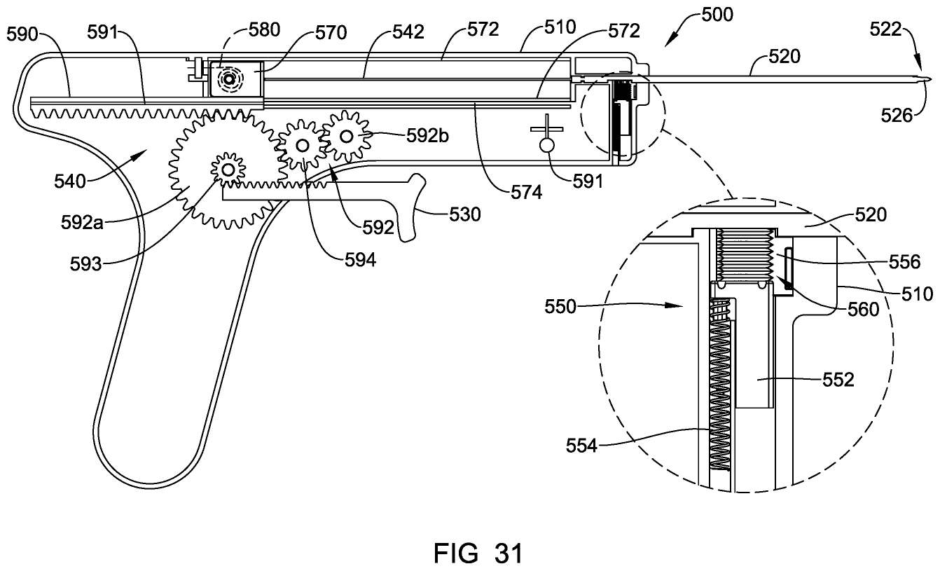

In some embodiments, the method of securing the implant 12 to tissue in the operative space in the body of the patient may comprise removing the locking pin 514 and/or the plurality of locking pins (ref. 514) from the handle 510, the handle housing, and/or the tissue anchor magazine 550, as seen in FIGS. 20 and 31. After removing the locking pin 514 and/or the plurality of locking pins (ref. 514), the biasing element 554 and/or the pushing element 552 may be configured to bias and/or urge the plurality of tissue anchors 560 toward the elongate shaft 520. For example, once the locking pin 514 and/or the plurality of locking pins (ref. 514) has been removed, the biasing element 554 may extend and/or expand toward the elongate shaft 520, thereby pushing the pushing element 552 and the plurality of tissue anchors 560 disposed on an opposite side of the pushing element 552 from the biasing element 554 toward the elongate shaft 520.

In some embodiments, the method of securing the implant 12 to tissue in the operative space in the body of the patient may comprise actuating the tissue anchor advancement mechanism 540 to deploy at least one tissue anchor of the plurality of tissue anchors 560 out the distal end 522 of the elongate shaft 520 (one at a time) into the implant 12 and tissue within the operative space, as seen in FIGS. 21-23 and 32-33. In some embodiments, the method of securing the implant 12 to tissue in the operative space in the body of the patient may comprise actuating the tissue anchor advancement mechanism 540 to deploy only one tissue anchor 562 of the plurality of tissue anchors 560 at a time out the distal end 522 of the elongate shaft 520 into the implant 12 and tissue within the operative space.

In some embodiments, actuating the tissue anchor advancement mechanism 540 and/or the pusher 542 may comprise engaging and/or actuating the trigger 530 coupled to the handle 510, wherein actuating the trigger 530 causes actuation of the tissue anchor advancement mechanism 540. In some embodiments, actuating the tissue anchor advancement mechanism 540 may comprise applying a distal force of the trigger 530 (e.g., sliding the trigger 530 distally). In some embodiments, actuating the tissue anchor advancement mechanism 540 may comprise applying a proximal force of the trigger 530 (e.g., squeezing and/or pulling the trigger 530 proximally). In some embodiments, actuating the tissue anchor advancement mechanism 540 may comprise applying a force transverse to the longitudinal axis of the elongate shaft 520 to the trigger 530 (e.g., squeezing the trigger 530 toward and/or relative to the handle 510 and/or the handle housing). Other configurations are also contemplated.

In some embodiments, actuating the tissue anchor advancement mechanism 540 may comprise extending the pusher 542 distally with respect to the elongate shaft 520. In some embodiments, actuating the tissue anchor advancement mechanism 540 may comprise extending the pusher 542 distally within the elongate shaft 520. In some embodiments, actuating the tissue anchor advancement mechanism 540 may comprise extending the pusher 542 from the handle 510 distally into the elongate shaft 520. In some embodiments, actuating the tissue anchor advancement mechanism 540 may comprise engaging the distal face 544 of the pusher 542 against the only one tissue anchor 562 to advance the only one tissue anchor 562 from the tissue anchor magazine 550 into the elongate shaft 520 and/or toward the distal end 522 of the elongate shaft 520. In some embodiments, the bevel 546 may be configured to engage a subsequent tissue anchor 564 of the plurality of tissue anchors 560 and bias remaining tissue anchors of the plurality of tissue anchors 560 away from the only one tissue anchor 562 of the plurality of tissue anchors 560 and/or the elongate shaft 520 as the only one tissue anchor 562 of the plurality of tissue anchors 560 is advanced distally, as seen in FIG. 21.

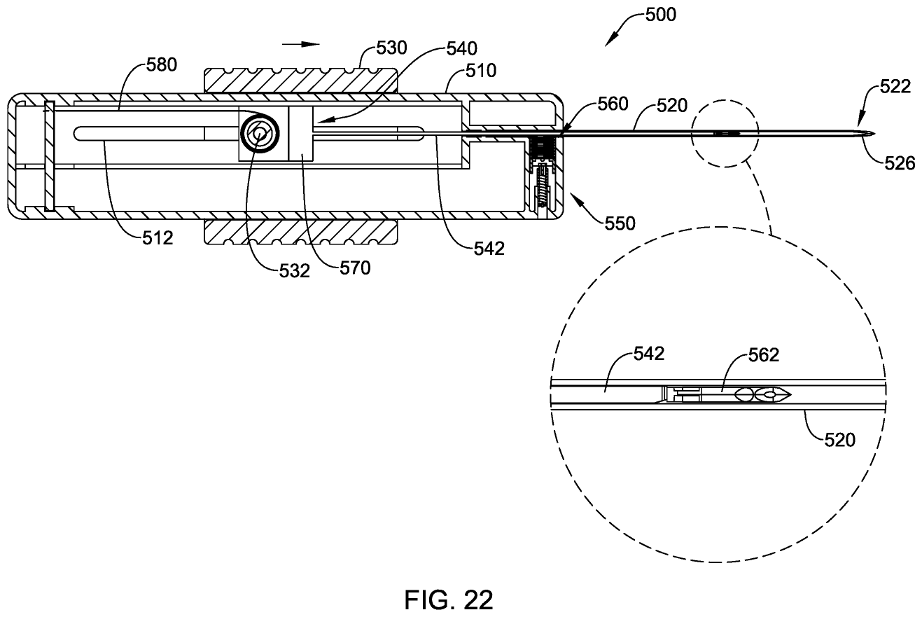

In some embodiments, the automatic retraction apparatus 580 may be configured to unwind and/or extend toward a distal end of the handle 510 as the slide block 570 and/or the pusher 542 is advanced distally and/or toward the distal end 522 of the elongate shaft 520 and/or toward the distal position, as seen in FIGS. 22-23 and 32-33.

In at least some embodiments, extending the pusher 542 may include extending the pusher 542 to the distal end 522 of the elongate shaft 520 to deploy the only one tissue anchor 562 into the implant 12 and/or tissue within the operative space, as seen in FIG. 23. In some embodiments, extending the pusher 542 may include extending the pusher 542 past and/or distal of the distal end 522 of the elongate shaft 520 to deploy the only one tissue anchor 562 into the implant 12 and/or tissue within the operative space. In some embodiments, when the slide block 570 is disposed in the distal position, the pusher 542 may extend to the distal end 522 of the elongate shaft 520. In some embodiments, when the slide block 570 is disposed in the distal position, the pusher 542 may extend past and/or distal of the distal end 522 of the elongate shaft 520. Other configurations are also contemplated.

In some embodiments, the method of securing the implant 12 to tissue in the operative space in the body of the patient may further comprise resetting the tissue anchor advancement mechanism 540. In some embodiments, resetting the tissue anchor advancement mechanism 540 may comprise resetting and/or translating the slide block 570 and/or the pusher 542 from the distal position to the proximal position.

In some embodiments, releasing pressure and/or distal force applied to the trigger 530 may permit the tissue anchor advancement mechanism 540 to reset and/or may permit the slide block 570 and/or the pusher 542 to reset and/or translate from the distal position to the proximal position. In some embodiments, the automatic retraction apparatus 580 may be configured to automatically reset the tissue anchor advancement mechanism 540 upon release of the trigger 530. In some embodiments, the automatic retraction apparatus 580 may be configured to automatically reset and/or translate the slide block 570 and/or the pusher 542 from the distal position to the proximal position upon release of the trigger 530.

In some embodiments, the tissue anchor advancement mechanism 540 may be configured to maintain a current position of the slide block 570 and/or the pusher 542 if and/or when the trigger 530 is released. In some embodiments, the tissue anchor advancement mechanism 540 may be actuated repeatedly in order to advance a tissue anchor of the plurality of tissue anchors 560 (e.g., the only one tissue anchor 562, etc.) along the entirety of the length of the elongate shaft 520. In some embodiments, a single actuation of the tissue anchor advancement mechanism 540 may advance a tissue anchor of the plurality of tissue anchors 560 (e.g., the only one tissue anchor 562, etc.) along the entirety of the length of the elongate shaft 520.

Thereafter, the method of securing the implant 12 to tissue in the operative space in the body of the patient may further comprise actuating the tissue anchor advancement mechanism 540 to deploy the subsequent tissue anchor 564 of the plurality of tissue anchors 560 out the distal end 522 of the elongate shaft 520 into the implant 12 and tissue within the operative space.

FIGS. 24-33 illustrated selected additional and/or alternative aspects of the tissue anchor delivery device 500 of FIGS. 18-23. It shall be understood that any aspect described herein may be used in conjunction with (e.g., in addition to) and/or in place of (e.g., may be exchanged with) any other aspect unless clearly stated to the contrary.

FIGS. 24-25 illustrate selected aspects of alternative configurations for the trigger 530. In some embodiments, the trigger 530 may be pivotally coupled to the handle 510. In some embodiments, the trigger 530 may be designed and configured such that a force transverse to the longitudinal axis of the elongate shaft 520 may be applied to the trigger 530 (e.g., squeezing the trigger 530 toward and/or relative to the handle 510 and/or the handle housing), as in FIG. 24. In some embodiments, the trigger 530 may be designed and configured such that a proximal force may be applied to the trigger 530 (e.g., squeezing and/or pulling the trigger 530 proximally), as in FIG. 25. In one example, the handle 510 may include a pistol-type grip. Other configurations are also contemplated.

FIGS. 24-25 also illustrate selected aspects of alternative configurations for the tissue anchor magazine 550. In some embodiments, the tissue anchor magazine 550 may be configured to be removably received within a receptacle 558 formed in the handle 510. In some embodiments, the pushing element 552 (not shown) and/or the biasing element 554 may be disposed within the tissue anchor magazine 550 as a removable assembly. In some embodiments, the removable assembly may further comprise the plurality of tissue anchors 560 disposed within the tissue anchor magazine 550. In at least some embodiments, the tissue anchor magazine 550 may be slidably received within the receptacle 558. In some embodiments, the tissue anchor magazine 550 may be replaceable within the handle 510 during a procedure and/or while the distal end 522 of the elongate shaft 520 is disposed in situ.

In some embodiments, the handle 510 and/or the tissue anchor magazine 550 may comprise a locking feature configured to prevent accidental removal of the tissue anchor magazine 550 from the receptacle 558. In some embodiments, the locking feature may comprise a catch mechanism (e.g., a hook, a pin, a ratchet, etc.) and a release element (e.g., a button, a slider, etc.) configured to disengage the catch mechanism. Other configurations are also contemplated.

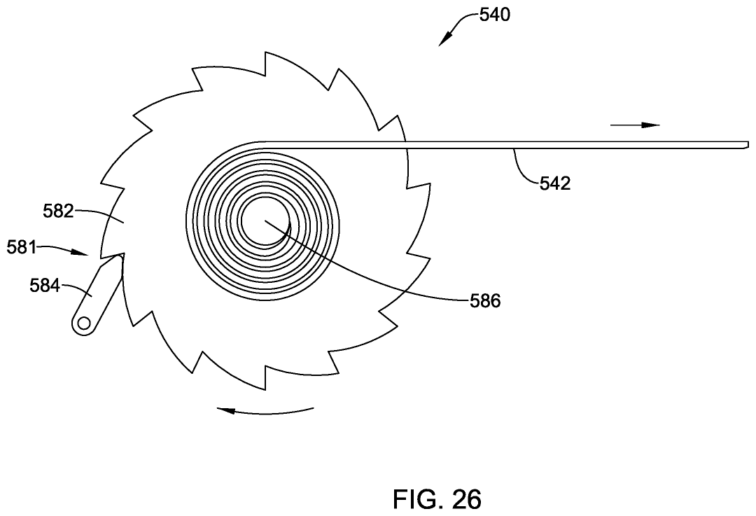

FIGS. 26-28 illustrate selected aspects of alternative configurations for the tissue anchor advancement mechanism 540. In some embodiments, the tissue anchor advancement mechanism 540 may comprise a ratcheting feature 581 disposed within the handle 510 (not shown). One example configuration of the ratcheting feature 581 is shown in FIG. 26. In some embodiments, the tissue anchor advancement mechanism 540 may comprise the pusher 542, wherein the pusher 542 or at least a portion of the pusher 542 may be coiled within the handle 510, such as around an axle 586 or an axis of rotation of the at least a portion of the pusher 542 coiled within the handle 510. In some embodiments, the tissue anchor advancement mechanism 540 and/or the axle 586 may be operatively coupled to the trigger 530 such that actuation of the trigger 530 causes the axle 586 to rotate and thereby uncoil and/or unwind the pusher 542.

The pusher 542 may be configured to uncoil and/or unwind as a tissue anchor of the plurality of tissue anchors 560 (e.g., the only one tissue anchor 562, etc.) is advanced along the length of the elongate shaft 520. As the pusher 542 is uncoiled and/or unwound, a distal end and/or a distal portion of the pusher 542 may be advanced into and/or along the length of the elongate shaft 520 to advance a tissue anchor of the plurality of tissue anchors 560 (e.g., the only one tissue anchor 562, etc.) along the length of the elongate shaft 520.

In some embodiments, the ratcheting feature 581 may comprise a gear 582 and a pawl 584 configured to engage with the gear 582 to prevent reverse rotation of the gear 582. In some embodiments, the ratcheting feature 581 may permit advancement of a tissue anchor of the plurality of tissue anchors 560 (e.g., the only one tissue anchor 562, etc.) along only a portion of the length of the elongate shaft 520 with each actuation of the tissue anchor advancement mechanism 540. In some embodiments, the ratcheting feature 581 may further comprise a release (not shown) configured to permit reverse rotation of the gear 582 and/or resetting of the tissue anchor advancement mechanism 540.

In some alternative configurations, the ratcheting feature may comprise a plurality of teeth formed in at least a portion of the pusher 542 and a pawl configured to engage the plurality of teeth formed in at least a portion of the pusher 542 as the pusher 542 is advanced into and/or along the length of the elongate shaft 520. The pawl may be configured to prevent proximal translation of the pusher 542. Other configurations are also contemplated.

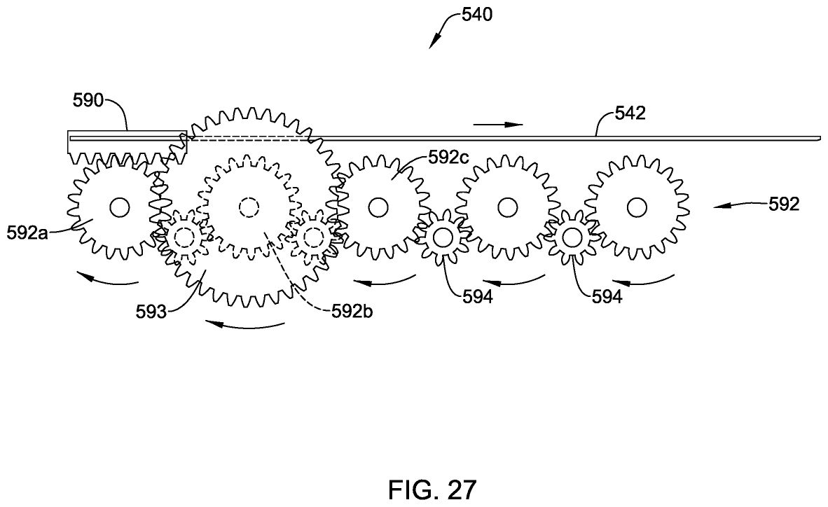

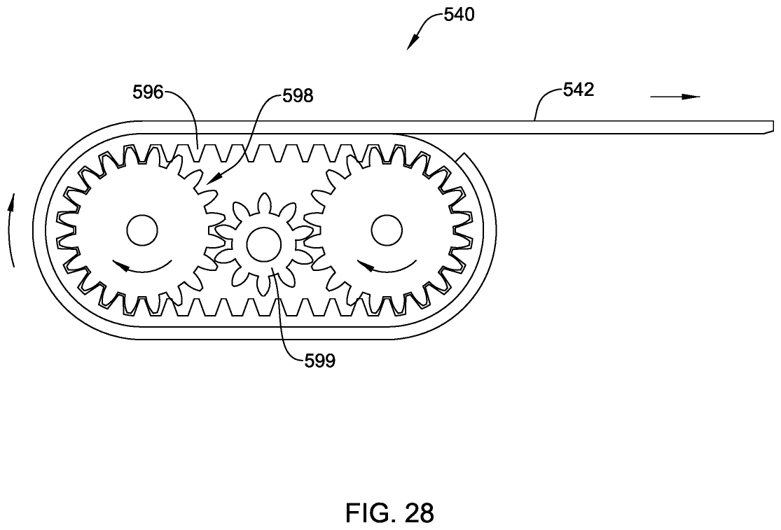

In some embodiments, the tissue anchor advancement mechanism 540 may comprise a plurality of gears 592 disposed within the handle 510 (not shown in FIGS. 27-28; shown in FIGS. 30-33). The plurality of gears 592 may be arranged longitudinally relative to each other within the handle 510, as seen in FIGS. 27 and 30-33. In some embodiments, the tissue anchor advancement mechanism 540 may comprise a linear rack 590 engaged with the plurality of gears 592. The pusher 542 may be coupled to and/or may be fixedly attached to the linear rack 590 and extend longitudinally therefrom. The plurality of gears 592 may be configured to translate the pusher 542 relative to the elongate shaft 520.

In some embodiments, at least one of the plurality of gears 592 may be operatively coupled to the trigger 530 such that actuation of the trigger 530 causes the plurality of gears 592 to rotate and thereby advance the pusher 542 into and/or along the length of the elongate shaft 520. In some embodiments, the linear rack 590 may be configured to engage multiple gears of the plurality of gears 592 as a tissue anchor of the plurality of tissue anchors 560 (e.g., the only one tissue anchor 562, etc.) is advanced along the length of the elongate shaft 520.

For example, as may be understood from FIGS. 27 and 30-33, as the plurality of gears 592 rotates, the linear rack 590 may translate distally, toward the distal end of the handle 510, and/or toward the elongate shaft 520. In some embodiments, the handle 510 may comprise a slot 574 configured to engage with a projection 591 extending laterally from the linear rack 590, as seen in FIGS. 30-33. The projection 591 may be configured to engage with and/or slide within the slot 574. In some embodiments, the handle 510 may comprise slots on opposing side of the linear rack 590 (e.g., one slot in each half of the handle 510, etc.) and the linear rack 590 may comprise projections extending laterally from opposing sides of the linear rack 590, wherein the projections are configured to engage with and/or slide within the slots. In some embodiments, the linear rack 590 may engage with a first gear 592a until the linear rack 590 engages a second gear 592b as a tissue anchor of the plurality of tissue anchors 560 (e.g., the only one tissue anchor 562, etc.) is advanced along the length of the elongate shaft 520. In some embodiments, the linear rack 590 may engage with the first gear 592a and the second gear 592b simultaneously as a tissue anchor of the plurality of tissue anchors 560 (e.g., the only one tissue anchor 562, etc.) is advanced along the length of the elongate shaft 520. The linear rack 590 may then engage with the second gear 592b until the linear rack 590 engages a third gear 592c as a tissue anchor of the plurality of tissue anchors 560 (e.g., the only one tissue anchor 562, etc.) is advanced along the length of the elongate shaft 520. In some embodiments, the linear rack 590 may engage with the second gear 592b and the third gear 592c simultaneously as a tissue anchor of the plurality of tissue anchors 560 (e.g., the only one tissue anchor 562, etc.) is advanced along the length of the elongate shaft 520. The arrangement and/or process may be repeated for as many gears as are provided in the plurality of gears 592 as a tissue anchor of the plurality of tissue anchors 560 (e.g., the only one tissue anchor 562, etc.) is advanced along the length of the elongate shaft 520.

In some embodiments, the plurality of gears 592 may comprise a drive gear 593 configured to engage with the trigger 530 and remaining gears to the plurality of gears 592. In some embodiments, the drive gear 593 may be coaxial with at least one other gear of the plurality of gears 592. In some embodiments, the drive gear 593 may be omitted and the trigger 530 may directly engage one of the plurality of gears 592 (e.g., the first gear 592a, the second gear 592b, the third gear 592c, etc. In some embodiments, the tissue anchor advancement mechanism 540 and/or the plurality of gears 592 may further comprise linking gears 594 (e.g., a linking gear, a plurality of linking gears, etc.) disposed between longitudinally adjacent gears of the plurality of gears 592 (e.g., between the first gear 592a and the second gear 592b, between the second gear 592b and the third gear 592c, etc.). In at least some embodiments, an axis of rotation of each linking gear of the linking gears 594 and an axis of rotation of each gear of the plurality of gears 592 (except for an axis of rotation of the drive gear 593, where present, which may be coaxial with another gear of the plurality of gears 592) may be longitudinally spaced apart from one another within the handle 510. Other configurations are also contemplated.

In some embodiments, the tissue anchor advancement mechanism 540 may comprise a continuously looped element 596 engaged with a plurality of gears 598 disposed within the handle 510. Similar to the plurality of gears 592 above, the plurality of gears 598 may be arranged longitudinally relative to each other within the handle 510, as seen in FIG. 28. The continuously looped element 596 may be disposed about the plurality of gears 598. The pusher 542 may be coupled to and/or may be fixedly attached to the continuously looped element 596. The plurality of gears 598 may be configured to translate the pusher 542 relative to the elongate shaft 520. In at least some embodiments, movement of the continuously looped element 596 about the plurality of gears 598 may be configured to translate the pusher 542 relative to the elongate shaft 520.

In some embodiments, at least a portion of the pusher 542 may be configured to at least partially wrap around the continuously looped element 596 prior to advancing the pusher into and/or along the elongate shaft 520, and/or prior to advancing a tissue anchor of the plurality of tissue anchors 560 (e.g., the only one tissue anchor 562, etc.) along the length of the elongate shaft 520. At least a portion of the pusher 542 may be configured to extend away from the continuously looped element 596 after advancing the pusher into and/or along the elongate shaft 520, and/or after advancing a tissue anchor of the plurality of tissue anchors 560 (e.g., the only one tissue anchor 562, etc.) along the length of the elongate shaft 520.

In some embodiments, the tissue anchor advancement mechanism 540 and/or the plurality of gears 598 may further comprise one or more linking gears 599 disposed between longitudinally adjacent gears of the plurality of gears 598. In at least some embodiments, an axis of rotation of each linking gear of the one or more linking gears 599 and an axis of rotation of each gear of the plurality of gears 598 (except for an axis of rotation of a separate drive gear, where present, which may be coaxial with another gear of the plurality of gears 598) may be longitudinally spaced apart from one another within the handle 510. Other configurations are also contemplated.

In some embodiments, the tissue anchor magazine 550 may comprise a tissue anchor guide 556, as seen in FIGS. 30-33. In at least some embodiments, each tissue anchor of the plurality of tissue anchors 560 may comprise a first leg and a second leg connected by a bridge portion. Within the tissue anchor magazine 550, the first leg and the second leg of each tissue anchor may be disposed on opposite sides of the tissue anchor guide 556. In some embodiments, the tissue anchor guide 556 may comprise at least one tissue anchor projection 557 (e.g., FIG. 30) extending laterally outward therefrom. In some embodiments, the at least one tissue anchor projection 557 may comprise a first tissue anchor projection extending from a first side of the tissue anchor guide 556 and a second tissue anchor projection extending from a second side of the tissue anchor guide 556, wherein the second side is opposite the first side. In some embodiments, the at least one tissue anchor projection 557 may comprise a plurality of first tissue anchor projections extending from the first side of the tissue anchor guide 556 and a plurality of second tissue anchor projections extending from the second side of the tissue anchor guide 556. Other configurations are also contemplated.

In some embodiments, the first tissue anchor projection(s) may be configured to slidably engage the first leg of each tissue anchor and the second tissue anchor projection(s) may be configured to slidably engage the second leg of each tissue anchor. The bridge portion of each tissue anchor may be configured to extend laterally with respect to the elongate shaft and/or to straddle a proximal edge, a proximal side, or a proximal portion of the tissue anchor guide 556.

The tissue anchor guide 556 may be configured to hold the plurality of tissue anchors 560 during assembly of the tissue anchor delivery device 500 and/or the handle 510. In one non-limiting example, the plurality of tissue anchors 560 may be loaded onto the tissue anchor guide 556, and subsequently, the tissue anchor guide 556 and the plurality of tissue anchors 560 disposed thereon may be loaded into the tissue anchor magazine 550 and/or the handle 510.

In some embodiments, the tissue anchor guide 556 may be configured to guide the plurality of tissue anchors 560 into the elongate shaft 520 and/or into an opening through a side of the elongate shaft 520 in a smooth and/or uniform manner. In some embodiments, the tissue anchor guide 556 may be configured to center the plurality of tissue anchors 560 with respect to the opening through the side of the elongate shaft 520. In some embodiments, the tissue anchor guide 556 may be configured to prevent damage to the distal tips of the plurality of tissue anchors 560 by preventing subsequent tissue anchors of the plurality of tissue anchors 560 from being pushed into an edge of the opening through the side of the elongate shaft 520 when a tissue anchor of the plurality of tissue anchors 560 (e.g., the only one tissue anchor 562, etc.) is advanced distally along the elongate shaft 520.

In some embodiments, the handle 510 and/or the handle housing may comprise an audible engagement element 591 (e.g., a clicker) configured to engage the tissue anchor advancement mechanism 540 and/or the linear rack 590 when the slide block 570 reaches the distal position and/or when a tissue anchor of the plurality of tissue anchors 560 (e.g., the only one tissue anchor 562, etc.) is deployed out the distal end 522 of the elongate shaft 520, as seen in FIG. 33. In some embodiments, the audible engagement element 591 (e.g., the clicker) may be configured to emit an audible sound (e.g., a click) when the audible engagement element 591 (e.g., the clicker) engages with the tissue anchor advancement mechanism 540 and/or the linear rack 590. In some embodiments, the distal end 522 of the elongate shaft 520 and/or the treatment site may not be readily visible when the tissue anchor of the plurality of tissue anchors 560 (e.g., the only one tissue anchor 562, etc.) is deployed, therefore the audible sound (e.g., the click) may be provided as a communication to the user that the tissue anchor of the plurality of tissue anchors 560 (e.g., the only one tissue anchor 562, etc.) has been deployed.

The materials that can be used for the various components of the medical devices, systems, and various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the tissue anchor(s), the tissue anchor delivery device, the elongate shaft, the tissue anchor advancement mechanism, etc., and/or elements or components thereof.

In some embodiments, the system and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN®), polyether block ester, polyurethane, polypropylene (PP), polyvinylchloride (PVC), polyether-ester (for example, ARNITEL®), ether or ester based copolymers (for example, butylene/poly (alkylene ether) phthalate and/or other polyester elastomers such as HYTREL®), polyamide (for example, DURETHAN® or CRISTAMID®), elastomeric polyamides, block polyamide/ethers, polyether block amide (PEBA, for example PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, portions or all of the system and/or components thereof may be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids a user in determining the location of the system. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-NR and the like), nitinol, and the like, and others.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-protein and/or anti-bacterial agents (such as 2-methacryroyloxyethyl phosphorylcholine (MPC) and its polymers or copolymers); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A tissue anchor delivery device, comprising:
a handle;
an elongate shaft extending distally from the handle, the elongate shaft having a length from the handle to a distal end of the elongate shaft of at least 10 cm;
a tissue anchor magazine coupled to the handle in communication with the elongate shaft, the tissue anchor magazine having a plurality of tissue anchors disposed therein; and
a tissue anchor advancement mechanism configured to advance the plurality of tissue anchors from the tissue anchor magazine out the distal end of the elongate shaft;
wherein the tissue anchor advancement mechanism is configured to advance only one tissue anchor of the plurality of tissue anchors along the length of the elongate shaft at a time.

2. The tissue anchor delivery device of claim 1, wherein each actuation of the tissue anchor advancement mechanism advances the only one tissue anchor of the plurality of tissue anchors only a portion of the length of the elongate shaft.

3. The tissue anchor delivery device of claim 1, wherein the tissue anchor advancement mechanism includes a ratcheting feature.

4. The tissue anchor delivery device of claim 1, wherein the tissue anchor advancement mechanism comprises a pusher coiled within the handle, the pusher being configured to advance the only one tissue anchor of the plurality of tissue anchors along the length of the elongate shaft.

5. The tissue anchor delivery device of claim 4, wherein the pusher is configured to uncoil as the only one tissue anchor of the plurality of tissue anchors is advanced along the length of the elongate shaft.

6. The tissue anchor delivery device of claim 1, wherein the tissue anchor advancement mechanism comprises a plurality of gears disposed within the handle, the plurality of gears being arranged longitudinally relative to each other within the handle.

7. The tissue anchor delivery device of claim 6, wherein the tissue anchor advancement mechanism comprises a continuously looped element engaged with the plurality of gears and a pusher coupled to the continuously looped element, the pusher being configured to advance the only one tissue anchor of the plurality of tissue anchors along the length of the elongate shaft.

8. The tissue anchor delivery device of claim 7, wherein at least a portion of the pusher is configured to at least partially wrap around the continuously looped element prior to advancing the only one tissue anchor of the plurality of tissue anchors along the length of the elongate shaft and configured to extend away from the continuously looped element after advancing the only one tissue anchor of the plurality of tissue anchors along the length of the elongate shaft.

9. The tissue anchor delivery device of claim 6, wherein the tissue anchor advancement mechanism comprises a linear rack engaged with the plurality of gears and a pusher coupled to the linear rack and extending longitudinally therefrom, the pusher being configured to advance the only one tissue anchor of the plurality of tissue anchors along the length of the elongate shaft.

10. The tissue anchor delivery device of claim 9, wherein the linear rack is configured to engage multiple different gears of the plurality of gears as the only one tissue anchor of the plurality of tissue anchors is advanced along the length of the elongate shaft.

11. The tissue anchor delivery device of claim 1, wherein the tissue anchor magazine is integrally formed with the handle.

12. The tissue anchor delivery device of claim 1, wherein the tissue anchor magazine is configured to be removably received within a receptacle formed in the handle.

13. The tissue anchor delivery device of claim 1, wherein the handle comprises a trigger configured to actuate the tissue anchor advancement mechanism.

14. The tissue anchor delivery device of claim 13, wherein the trigger is pivotably coupled to the handle.

15. The tissue anchor delivery device of claim 13, wherein the trigger is slidably coupled to the handle.

16. The tissue anchor delivery device of claim 1, wherein the tissue anchor advancement mechanism comprises an automatic retraction apparatus configured to reset the tissue anchor advancement mechanism.

17. The tissue anchor delivery device of claim 1, wherein the tissue anchor advancement mechanism comprises a pusher configured to engage the only one tissue anchor of the plurality of tissue anchors;
wherein the pusher comprises a distal face and a bevel extending proximally from the distal face to a bottom surface such that a height of the distal face is less than a thickness of the only one tissue anchor of the plurality of tissue anchors.

18. The tissue anchor delivery device of claim 17, wherein the bevel is configured to engage a subsequent tissue anchor of the plurality of tissue anchors and bias remaining tissue anchors of the plurality of tissue anchors away from the only one tissue anchor of the plurality of tissue anchors as the only one tissue anchor of the plurality of tissue anchors is advanced distally.

19. A method of securing an implant to tissue in an operative space in a body of a patient, comprising:
advancing the implant into the operative space within an elongate shaft of an implant delivery device;
deploying the implant adjacent tissue within the operative space;
inserting a tissue anchor delivery device into the operative space, the tissue anchor delivery device comprising:
a handle;
an elongate shaft extending distally from the handle, the elongate shaft having a length from the handle to a distal end of the elongate shaft of at least 10 cm;
a tissue anchor magazine coupled to the handle in communication with the elongate shaft, the tissue anchor magazine having a plurality of tissue anchors disposed therein; and
a tissue anchor advancement mechanism configured to advance the plurality of tissue anchors from the tissue anchor magazine out the distal end of the elongate shaft;
actuating the tissue anchor advancement mechanism to deploy at least one tissue anchor of the plurality of tissue anchors out the distal end of the elongate shaft into the implant and tissue within the operative space;
wherein the tissue anchor advancement mechanism is configured to advance only one tissue anchor of the plurality of tissue anchors along the length of the elongate shaft at a time.

20. A system for delivering tissue anchors into an operative space in a body of a patient, comprising:
a tissue anchor delivery device comprising a handle and an elongate shaft extending distally from the handle; and
a tissue anchor magazine separate and detached from the tissue anchor delivery device, the tissue anchor magazine having a plurality of tissue anchors stored therein;
wherein the tissue anchor magazine is configured to be delivered subcutaneously into the operative space separate from the tissue anchor delivery device.
